(19) United States
(12) Patent Application Publication
(10) Pub. No.: US 2023/0256102 A1
(43) Pub. Date: Aug. 17, 2023

(54) COMPOSITES AND USES THEREOF

(71) Applicant: YISSUM RESEARCH DEVELOPMENT COMPANY OF THE HEBREW UNIVERSITY OF JERUSALEM LTD., Jerusalem (IL)

(72) Inventors: Zvi Hayouka, Mazkeret Batya (IL); Saul Burdman, Rehovot (IL); David Avnir, Ashkelon (IL); Barak Menagen, Jerusalem (IL); Aya Brill, Tel Aviv (IL); Tal Stern Bauer, Jerusalem (IL); Dafna Tamir Ariel, Karmey Yosef (IL); Heli Tamir Buchnik, Rehovot (IL); Shiri Topman, Ramat Gan (IL)

(21) Appl. No.: 17/620,467

(22) PCT Filed: Jun. 18, 2020
(86) PCT No.: PCT/IL2020/050681

§ 371 Date: Dec. 17, 2021

Related U.S. Application Data
(60) Provisional application No. 62/863,875, filed on Jun. 20, 2019.
(60) Provisional application No. 62/864,106, filed on Jun. 20, 2019.
(60) Provisional application No. 62/864,126, filed on Jun. 20, 2019.

(51) Int. Cl. A61K 47/54; A01P 1/00; A61P 31/04; A01N 37/44

(52) U.S. Cl. CPC A61K 47/542; A01P 1/00; A61P 31/04; A01N 37/44

(57) ABSTRACT

The invention generally concerns active peptide mixture and composites thereof.

### CROSS-REFERENCE TO RELATED APPLICATIONS

The present application is a National Phase Entry of PCT International Application No. PCT/IL2020/050681 which was filed on Jun. 18, 2020, which claims priority to U.S. Provisional Patent Application No. 62/863,875, filed Jun. 20, 2019, U.S. Provisional Patent Application No. 62/864,106, filed Jun. 20, 2019, and U.S. Provisional Patent Application No. 62/864,126, filed Jun. 20, 2019, all of which are hereby incorporated by reference in their entireties.

### TECHNOLOGICAL FIELD

The present invention generally concerns novel composites and uses thereof.

### BACKGROUND

The options to cope with bacterial diseases are limited and the available strategies for their management are often inadequate. There is an unmet need for new strategies for controlling or preventing bacterial infections without the use of antibiotics that can induce resistant species. Similarly, there is a growing need for antifungal agents that are based on ecofriendly molecules rather than chemical agents that are known to have adverse effects.

Fungi constitute the largest group of plant pathogens and are responsible for a range of severe plant diseases. Phytopathogenic fungi play a dominant role not only by causing devastating epidemics, but also through a less noticeable although persistent and significant annual crop-yield losses that have made fungal pathogens of plants a substantial economic factor.

Current treatments for fungi population control typically include chemicals, biologicals, and/or non-chemical methods such as methods aimed to provide resistant crop strains, GMOs, and inhibitors to clear loci prior to planting. Each of these chemical and biological classes of compounds and methods has one or more drawbacks, including, but not limited to, toxicity, cost, availability, and reliability.

Microbial plant pathogens can significantly reduce quality and yield of crops. It is estimated that about 10 to 15% of the agricultural production worldwide is lost to plant diseases caused by biotic agents, causing over USS 220 billion of economic damage every year. Plant pathogenic bacteria are among the most important causal agents of plant diseases with almost all major crops being severely affected by at least one, and often more bacterial diseases. Bacteria as plant pathogens can cause a variety of damaging diseases, ranging from spots, mosaic patterns or pustules on leaves and fruits, or smelly tuber rots to plant death. Some cause hormone-based distortion of leaves and shoots called fasciation, or crown gall, a proliferation of plant cells producing a swelling at the intersection of stem and soil and on roots.

Antimicrobial peptides (AMPs), also called host defense peptides (HDPs), are part of the innate immune response found among all classes of life including mammals, amphibians, insects, and plants. The mode of HDPs action varies among different cases, and a particular HDP may have more than one antimicrobial mechanism; however, many HDPs share the ability to disrupt microorganisms' membranes. HDPs display a characteristic selectivity, favoring attack on prokaryotic membranes relative to eukaryotic membranes. This selectivity is thought to arise from the net cationic charge common to HDPs, since the external surfaces of prokaryotic cells typically have a larger net negative charge than do the external surfaces of eukaryotic cells. HDPs are rich in hydrophobic residues, which presumably mediate disruptive interactions with the hydrophobic interior of a lipid bilayer. The broad molecular diversity among HDPs suggests that their prokaryotic-selective activity is not tightly coupled to specific features of amino acid sequence or peptide conformation.

Low concentrations of certain metals such as copper and silver, exhibit bactericidal properties and are thought to work either extracellularly, by binding and inactivating membrane proteins, or intracellularly, after transport into the cell. Although silver has stronger bactericidal activity, copper is less expensive and is therefore used more extensively as a safe, efficient, and inexpensive bactericide in the fields of health, microbial disease control, hygiene, and agriculture. Specific examples of copper use include water treatment facilities, disinfection of surfaces in the food industry, hospital sterilization, adding antifouling properties to paints, and wound healing. The metal is also commonly used on a large scale in agriculture for crop protection.

The microbial toxicity of copper is mediated through multifactorial pathways, one of which exploits the ability of copper to act as a catalyst for the generation of reactive oxygen species (ROS), which then cause oxidative damage to vital cell constituents such as proteins, lipids, DNA, and other biomolecules. Copper ions can also deactivate inner-cell and envelope proteins either by direct interaction or via competition for the binding sites of essential metals. In both cases, inhibition or deactivation of the protein is the result of conformational changes in the protein structure or active site.

Bacterial resistance to copper and other antibiotics has created an urgent need for the development of novel solutions to combat the resistant bacteria. This need has spurred studies of the antimicrobial or anti-biofilm activity exhibited by copper together with combinations of amino acids, organic acids, peptides such as hepcidine and the introduction of peptide sequences containing a copper binding motif.

Incorporation and entrapment of molecules in metals often results in new synergistic properties. Composites made by entrapping known antimicrobial agent, such as chlorohexidine in silver (CH@Ag), have presented synergistic antimicrobial activities [1,2]. When silver was replaced by copper, the composite presented even stronger antimicrobial properties [3]. Strong antibacterial activity was even observed by the incorporation of anti-inflammatory agents within silver composites [4-7].

Antimicrobial peptides (AMPs) represent a new approach to tackle pathogenic bacteria. They are natural substances produced by eukaryotes, composed of 10-50 amino acids and display strong antimicrobial activity against several pathogenic bacteria. The broad diversity of AMPs in terms of sequences and structures inspired us the development of random peptide mixtures (RPMs), which are composed of hydrophobic and cationic amino acids in binary ratio, to generate random sequences with controlled amino acid composition and chain length. These mixtures were found to possess potent antimicrobial activity against a broad spectrum of bacteria. Synthesis of RPMs is cheaper and simpler compared to specific peptides and enables to obtain mixture of 2²⁰ slightly different peptides, which will challenge the bacteria to acquire resistance [8,9].

### BACKGROUND ART

[1] R. Ben-Knaz, R. Pedahzur, D. Avnir, A concept in bactericidal materials: The entrapment of chlorhexidine within silver, Adv. Funct. Mater. 20 (2010) 2324-2329.
[2] R. Ben-Knaz, R. Pedahzur, D. Avnir, Bioactive doped metals: high synergism in the bactericidal activity of chlorhexidine@silver towards wound pathogenic bacteria, RSC Adv. 3 (2013) 8009.
[3] R. Ben-Knaz Wakshlak, R. Pedahzur, B. Menagen, D. Avnir, An antibacterial copper composite more bioactive than metallic silver, J. Mater. Chem. B. 4 (2016) 4322-4329.
[4] B. Menagen, R. Pedahzur, D. Avnir, Sustained release from a metal—Analgesics entrapped within biocidal silver, Sci. Rep. 7 (2017) 1-11.
[5] U.S. Pat. No. 9,643,168.
[6] International patent publication no. WO2011/135563.
[7] International patent publication no. WO2013/108259.
[8] Amso Z, Hayouka Z. Antimicrobial random peptide cocktails: a new approach to fight pathogenic bacteria. Chem Commun (Camb). 2019; 55, 2007-2014.
[9] Hayouka, Z. Chakraborty, S. Liu, R. Gellman, H. S. (2013). Interplay Among Subunit Identity, Composition, Chain Length and Stereochemistry in the Activity Profile of Sequence-Random Oligomer Mixtures. J. Am. Chem. Soc. 135(32):11748-51.

### GENERAL DESCRIPTION

Random antimicrobial peptide mixtures (RPMs), were originally inspired by the natural antimicrobial peptides (AMPs) produced by eukaryotes as part of their innate immune response to bacterial infection. AMPs are typically cationic and act primarily by forming electrostatic interactions with the anionic bacterial membrane, followed by insertion into the membrane bilayers where the hydrophobic residues of the AMPs cause membrane disruption and bacterial cell death. Despite the broad molecular diversity of natural AMPs in terms of sequences and structures, the inventors of the invention disclosed herein have identified a number of common features useful in designing random cationic peptide mixtures that possess strong antimicrobial and antibiofilm properties.

The RPMs are synthesized by mixing a defined ratio of a hydrophobic amino acid and a cationic amino acid, optionally also their enantiomers or other non-natural amino acids, to generate a mixture of peptides having a desired chain lengths, and having different peptide sequences and optionally different stereochemistries. These different sequences confer the ability to target a wide variety of bacterial groups with a low probability of developing bacterial resistance. Another great advantage of RPMs, is the low cost of synthesis as no purification step is needed.

The inventors of the present invention have also developed conjugates which comprise RPM peptides that are covalently associated to fatty moieties (e.g., derived from fatty acids).

Thus, in one of its aspects, the invention provides a mixture of peptides, each peptide being a 4-mer to 15-mer peptide having or constructed of at least one hydrophobic amino acid and/or at least one cationic amino acid, wherein at least a portion or each of the peptides is optionally covalently associated to a fatty moiety.

As disclosed herein, each of the peptides in a mixture of the invention comprises or consists between 4 and 15 amino acids that are selected amongst hydrophobic and cationic amino acids. The number of amino acids of each type may vary and in some cases is random. Each of the amino acids in the peptides is associated to another of the amino acids via a peptide bond, as known in the art. In some embodiments, the amino acids making up a peptide in a mixture of the invention are all hydrophobic amino acids or all cationic amino acids. In other words, in such embodiments, the peptide consists of hydrophobic amino acids or cationic amino acids. In other embodiments, the peptide in a mixture of the invention may comprise a random combination of hydrophobic amino acids and cationic amino acids.

The peptides may comprise L-amino acids, D-amino acids (e.g., D-Lys), or combinations thereof. The amino acids may be selected from natural and non-natural amino acids. Peptides having both D-amino acid residues and L-amino acid residues are defined herein as diastereomeric peptides. In some embodiments, the peptide comprises all L-amino acids. In some other embodiments, the peptide comprises all D-amino acid. In further embodiments, the peptide is selected amongst diastereomeric peptides.

The selection of amino acids may dictate the number of peptides in a mixture of the invention. For example, the number of peptides in a mixture may be at least 2ⁿ (wherein n is the length of the peptide), where two amino acids are used. The number of peptides may be 3ⁿ in cases where one of the two amino acids is in the D- or L-form, and at least 4ⁿ in cases where both amino acids are in the L- and D-forms.

In some embodiments, the peptides comprise natural or non-natural amino acids. In some embodiments, a mixture of the invention further comprises peptides constructed of non-natural amino acids.

In some embodiments, the peptides comprise amino acid(s) selected from β-amino acids, γ-amino acid, D-amino acid, cyclic peptides and any combination thereof.

In some embodiments, the peptide is a cyclic peptide.

In some embodiments, the peptide is a glycosylated peptide or an acetylated peptide.

In some embodiments, the number of amino acid residues in a peptide is at least 4 but no more than 30, inclusive. In some embodiments, the number of amino acids is between 4 and 15. In some embodiments, the number of amino acid residues is 5, or 6, or 7, or 8, or 9, or 10, or 11, or 12, or 13, or 14, or 15.

In some embodiments, the peptide comprises at least five amino acid residues. In some embodiments, the peptide consists of between 5 and 10 amino acid residues, 5 and 8 amino acid residues or 5 and 7 amino acid residues. In some embodiments, the peptide consists of between 15 and 30 amino acid residues, 15 and 25 amino acid residues or 20 and 17 amino acid residues.

The number of hydrophobic amino acids relative to the number of cationic amino acids (namely the ratio of the two), as well as the proximity of the hydrophobic amino acid to the fatty moiety, where present, can govern an increase in the antimicrobial effect of peptides of the invention. Peptides containing a greater number of hydrophobic amino acids as compared to the cationic amino acids exhibited lesser antibacterial activity (i.e., exhibited a higher minimum inhibitory concentration, MIC). For example, when comparing two mixtures of peptides, which include phenylalanine (Phe, F) and lysine (Lys, K), a mixture that contained a higher number of lysine amino acids, e.g., F:K at a 1:9 ratio, had a significantly lower MIC compared to a mixture that contained equal numbers of both amino acids, e.g., F:K at a ratio of 1:1. In other words, in some embodiments, mixtures containing a higher percentage of Lys exhibited better antimicrobial activity.

To further demonstrate the antimicrobial effect of peptide mixtures of the invention, a 5-mer peptide will be described herein using the formula XXXXX, wherein each X designates an amino acid making up the peptide. The peptide comprises or consists a cationic amino acid, designated with the letter C, and/or a hydrophobic amino acid, designated with the letter H.

The peptide may be constructed of amino acid C, wherein all or some of variants X is C (and the other of the variants X is H) or wherein all or some of variants X is H (and the other of the variants X is C). Where #C>#H (the number of C is greater than the number of H), the MIC is lower, thus exhibiting higher antibacterial activity. In contrast, in other embodiments, where #C<#H, the MIC is higher, thus exhibiting a lower antibacterial effect.

Thus, in some embodiments, the ratio between C:H is at least 1:1. In some embodiments, the ratio C:H is at least 2:1. In some embodiments, the ratio is at least 3:1. In some embodiments, the ratio is at least 4:1. In some embodiments, the ratio is at least 5:1. In some embodiments, the ratio is at least 6:1. In some embodiments, the ratio is at least 7:1. In some embodiments, the ratio is at least 8:1. In some embodiments, the ratio is at least 9:1.

In some embodiments, the ratio C:H is between 1:1 and 10:1.

In some embodiments, the mixture comprising one or more of the peptides, designated herein as peptides (1) to (15):

(1) CCCCC, (2) CCCCH, (3) CCCHC, (4) CCHCC, (5) CHCCC, (6) HCCCC, (7) CCCHH, (8) CCHCH, (9) CHCCH, (10) HCCCH, (11) CCHHC, (12) CHCHC, (13) HCCHC, (14) CHHCC and (15) HCHCC. Other peptides in which the #C is greater than #H may also be comprises in a mixture of the invention.

In some embodiments, a mixture may comprise peptides constructed of 2 or more amino acids. In some embodiments, a mixture comprises 3 amino acids, at a ratio Phenylalanine (25%):Leucine (25%):Lysine (50%).

The “cationic amino acids” may be selected from amino acids having positively charged side chains, as known in the art, or any derivative thereof. Non-limiting examples include lysine, arginine, histidine, ornithine, di-amino butyric acid (Dab) and di amino propionic acid (Dap). In some embodiments, the cationic amino acid is selected from lysine, arginine and histidine. In some embodiments, the cationic amino acid is lysine or arginine.

The “hydrophobic amino acids” may be selected from the amino acids having low or no water solubility, or any derivative thereof. Non-limiting examples include proline, methionine, tryptophan, phenylalanine, leucine, isoleucine, glycine, alanine and valine. In some embodiments, the hydrophobic amino acid is selected from phenylalanine, leucine, isoleucine, glycine, alanine and valine. In some embodiments, the hydrophobic amino acid is phenylalanine.

In some embodiments, the peptide comprises or consists the amino acids phenylalanine and lysine.

In some embodiments, in peptides herein designated peptides (1) through (15) and other peptides of the formula XXXXX, the amino acid designated H is phenylalanine (F) and the amino acid designated C is lysine (K). Thus, in some embodiments, a mixture of the invention may comprise one or more of the following peptides: KKKKK, KKKKF, KKKFK, KKFKK, KFKKK, FKKKK, KKKFF, KKFKF, KFKKF, FKKKF, KKFFK, KFKFK, FKKFK, KFFKK and FKFKK.

In some embodiments, in a mixture of the invention, the peptides are constructed of identical amino acids (e.g., KKKKK). In some other embodiments, the peptides are random peptides, wherein each peptide has a sequence of amino acids that is different (e.g., some are KFKKK, some KKKKK, others are KKFFK, etc).

Where the peptide is provided in the form of a conjugate with a fatty moiety, the distance or rather proximity of the hydrophobic amino acid to the fatty moiety may also have an effect on the antibacterial activity. In some cases, for example, where the hydrophobic amino acid is in a proximate position to the fatty moiety, the antibacterial activity is lower. Where the two are further apart, the activity may increase. For example, a peptide of the formula p-HHXXX exhibits a reduced antibacterial activity compared to P-CXXXX.

The reduced activity demonstrated in cases where the number of hydrophobic amino acids is larger than the number of cationic amino acids in the peptide mixture (namely a ratio H:C is high), as defined above, and where the hydrophobic amino acid is in a proximate position in relation to the fatty moiety, where present, is believed to be due to aggregation of the peptides resulting from high lipophilic interactions.

Thus, in some embodiments of the invention, peptides in mixtures of the invention comprise at least one cationic amino acid and at least one hydrophobic amino acids, wherein the sum (number) of all cationic amino acids in the mixture is higher than the sum (number) of all hydrophobic amino acids in the mixtures.

Additionally, in some embodiments where peptides in mixtures of the invention are conjugated to fatty moieties, the fatty moieties are conjugated or associated to the peptide at a position that is further apart (e.g., at a maximal distance) from the at least one hydrophobic amino acid in the peptide(s).

Random mixtures, namely mixtures containing random peptides, as defined, such as a mixture randomly comprising any combination or a combination of all of the herein designated peptides (1) to (15) exhibits a higher antibacterial activity as compared to a mixture consisting any one of the peptides alone. This phenomenon occurs, without being bound by a theory, due to a synergistic effect resulting from the various types of peptides in the mixture.

In some embodiments, in a mixture of the invention, peptides of 4 to 15 amino acids in length that are optionally conjugated to a fatty moiety, are presented, wherein each of the peptides in the mixture consists of hydrophobic and/or cationic amino acids, wherein the ratio, in the mixture, of total hydrophobic amino acids to total cationic amino acids is between 3:1 and 1:3. In some embodiments, the ratio in a mixture of the total hydrophobic amino acids to the total cationic amino acids is between 2:1 and 1:2. In some embodiments, the ratio is about 1:1.

In some embodiments, in a mixture of the invention, the peptide consist of phenylalanine and/or lysine residues, wherein the ratio, in the mixture, of the total phenylalanine residues to the total lysine residues is between 3:1 and 1:3.

As used herein, the term “ratio”, as applied to the ratio amounts of amino acids within a mixture, refers to the ratio between different amino acid types, wherein each of the amino acid forms part of the peptides present in the mixture. For example “a ratio in the mixture of the total hydrophobic amino acids to the cationic amino acids” refers to the number of all hydrophobic amino acids forming part of the peptides in the mixture relative to the number of all cationic amino acids forming part of the peptides in the same mixture.

As stated herein, peptides used in mixtures of the invention may be conjugated or covalently associated to a fatty moiety that may be derived from a fatty acid. Such a conjugation results in a lipophilic conjugate which properties may be varied and tailored by, e.g., a particular site of conjugation, a particular amino acid for conjugation, by selecting a suitable fatty acid, etc. The fatty moiety may be derived from saturated, unsaturated, monounsaturated and polyunsaturated fatty acids, as known in the art. The fatty moiety may consist of at least eight carbon atoms, and thus may be selected and derived from such fatty acids as decanoic acid, undecanoic acid, dodecanoic acid (lauric acid), myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, cerebronic acid and others.

The fatty moiety may be coupled to the N-terminal, to the C-terminal, or to any other free functional group along the peptide chain. As such, the moiety may be derived from any precursor molecule that upon reaction therewith results in covalent association of the fatty moiety with the atom or group on the peptide chain. The fatty moiety may be thus be associated to the peptide (or an amino acid of the peptide) via any covalent functionality such as via an ester group, an amide group, an amine group, an oxo group, a thio group, and others.

In some embodiments, the fatty acid may be conjugated to the ε-amino group of lysine.

In some embodiments, the fatty moiety is derived from palmitic acid or lauryl acid.

As used herein, a “mixture” of the invention is a mixture or a combination of peptides which comprises two or more peptides that have the same length (in terms of number of amino acids in the peptide), but differ in their amino acid sequence and optionally also in their stereochemistry. Thus, for example, a mixture of the invention may comprise a plurality or two or more peptides, each comprising 5 amino acids (a 5-mer peptide), being leucine and lysine, wherein the two or more peptides differ in the sequence of leucine and lysine. Where a peptide is conjugated to a fatty moiety, the mixture may contain conjugated and non-conjugated peptides.

A mixture of the invention may be presented in a form of a solid mixture, a dispersion (e.g., in water or an aqueous buffer) or as a solution (e.g., in water or an aqueous buffer) and the peptides may be presented in neutral or charged form.

Mixtures of the invention provide a low cost and powerful antimicrobial tool that is highly efficient against bacterial and fungal infections and diseases. Advantageously, while presenting a significant antimicrobial activity, mixtures of the invention also reduce microorganisms' ability to develop resistance. In some cases, conjugation of fatty moieties to the peptides can significantly enhance the mixtures' antimicrobial activity.

The “antimicrobial activity” is intended to encompass inhibition, prevention or destruction of growth or proliferation of microorganisms such as bacteria, fungi, protozoa, viruses, molds and the like. Thus, in some embodiments, the mixture has an antimicrobial activity; in some embodiments, an antibacterial activity; in some embodiments, an antifungal activity; and in other embodiments, the mixture of the invention has a combined activity, namely is active against two or more microorganisms of the same or different class.

Mixtures of the invention may be used to prepare compositions having antimicrobial activities. These compositions may be pharmaceutical compositions or formulations or compositions for general use.

Thus, in another one of its aspects, the invention provides a composition comprising a mixture of the invention. In some embodiments, the composition comprises an amount of the mixture that is sufficient to exert an antimicrobial activity. In some embodiments, this effective amount may vary based on the composition of the invention, its intended use and mode of application. The “effective amount” is an amount sufficient to achieve a beneficial or a desired result. An effective amount can be administered in one or more different modes of administrations. In terms of treatment and prevention, the effective amount is that amount sufficient to palliate, ameliorate, stabilize, reverse, slow or delay the progression of fungal or bacterial disease state.

In some embodiments, the composition comprises at least 6 μg/mL of the mixture. In some embodiments, the composition comprises between 3 and 20 μg/mL of the mixture and in other embodiments, 10 and 14 μg/mL of the mixture.

In some embodiments, the composition is used or administered in a mixture concentration that inhibits growth of at least 40% of the bacteria or fungi. In other embodiments, the composition is used or administered in a mixture concentration that inhibits the growth of at least 50% of the bacteria or fungi, or least 60%, or at least 70%, or at least 80%, or at least 90%, or at least 95%, or at least 96%, or at least 97%, or at least 98%, or at least 99% or 100% of the bacteria or fungi.

In some cases, the activity, as defined herein, may be increased by conjugating the peptide(s) to a fatty moiety. In such embodiments, the antimicrobial activity of a mixture of conjugated peptides may be at least 2, 4, 6, 8, 10, 12, 14, or 15 times higher than that of the same peptides in free or unconjugated form. In some cases, the antimicrobial activity of the mixture is at least 10 times higher or at least 15 times higher than that of the same peptides in an unconjugated form.

In some embodiments, the microorganism is a bacterium or fungus.

In some embodiment, the microorganism is a antibiotic-resistant bacterium.

In some embodiments, the bacterium is selected from Gram-negative and Gram-positive bacteria.

In some embodiments, the bacterium is selected from those known in the art. In some embodiments, the microorganism is selected from Bacillus anthracis; Clostridium botulinum; Francisella tularensis; Yersinia pestis; Burkholderia pseudomallei; Burkholderia mallei; Clostridium perfringens; Coxiella burnetii; Brucella melitensis, abortus, suis and canis; Staphylococcus aureus; Rickettsia prowazekii; Chlamydia psittaci; Food and Waterborne Pathogens such as Escherichia coli, Vibrio cholerae, Salmonella species, Shigella species, Listeria monocytogenes, Campylobacter jejuni, Yersinia enterocolitica; Mycobacterium tuberculosis; and other Rickettsia.

In some embodiments, the microorganism is methicillin-resistant Staphylococcus aureus (MRSA), vancomycin-resistant enterococci (VRE), Clostridium difficile, Acinetobacter baumannii and multi-drug resistant (MDR) Acinetobacter sp.

In some embodiments, the microorganism is selected from Xanthomonas, Agrobacterium, Erwinia, Leifsonia, Pectobacterium, Pseudomonas, Ralstonia, and Xylella. In some embodiments, the bacterium is selected from Xanthomonas campestris pv. campestris (Xcc) and Xanthomonas campestris pv. vesicatora (Xcv).

In some embodiments, the fungus is selected from Candida, Aspergillus, Cryptococcus, Histoplasma, Pneumocystis and Stachybotrys.

In some embodiments, the fungus is selected from Candida albicans, Candida amphixiae, Candida antarctica, Candida argentea, Candida ascalaphidarum, Candida atlantica, Candida atmosphaerica, Candida auris, Candida blankie, Candida blattae, Candida bracarensis, Candida bromeliacearum, Candida carpophila, Candida carvajalis, Candida cerambycidarum, Candida chauliodes, Candida corydalis, Candida dosseyi, Candida dubliniensis, Candida ergatensis, Candida fermentati, Candida fructus, Candida glabrata, Candida guilliermondii, Candida haemulonii, Candida humilis, Candida insectamens, Candida insectorum, Candida intermedia, Candida jeffresii, Candida kefyr, Candida keroseneae, Candida krusei, Candida lusitaniae, Candida lyxosophila, Candida maltose, Candida marina, Candida membranifaciens, Candida mogii, Candida oleophila, Candida oregonensis, Candida parapsilosis, Candida quercitrusa, Candida rhizophoriensis, Candida rugosa, Candida sake, Candida sharkiensis, Aspergillus fumigatus, Aspergillus flavus, Cryptococcus neoformans, Cryptococcus laurentii, Cryptococcus albidus, Cryptococcus gattii, Histoplasma capsulatum, Pneumocystis jirovecii (or Pneumocystis carinii) and Stachybotrys chartarum.

In some embodiments, the fungus is Candida albicans.

In some embodiments, the microorganisms are plant related.

In some embodiments, the bacteria are selected from Gram-negative and Gram-positive plant-pathogenic bacteria.

In some embodiments, the plant disease is caused by bacteria, e.g., antibiotic-resistant bacteria. In some embodiments, the bacteria is of a genera selected from Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma.

In some embodiments, the bacteria are selected from Burkholderia, and Pseudomonas syringae. In some embodiments, the bacteria are selected from Xanthomonas spp. such as Xanthomonas campestris pv. campestris (Xcc) and Xanthomonas campestris pv. vesicatora (Xcv).

In some embodiments, the bacteria are selected from Pseudomonas spp.

In some embodiments, mixture of the invention are useful in inhibiting growth of strains of Clavibacter michiganensis subsp. michiganensis (bacterial canker and wilt of tomato), Xanthomonas perforans (bacterial spot disease of tomato and pepper), Xanthomonas campestris pv. campestris (black rot disease of Brassicaceae plants) and Acidovorax citrulli (bacterial fruit blotch of cucurbit plants). The variety of diseases that are caused by pathogens such as bacteria and fungi that are affected by mixtures of the invention are rather vast. Diseases that are thus treatable or manageable by mixtures or compositions of the invention are any one or more disease that is, directly or indirectly, caused by any of the pathogens. A partial list of such diseases is provided in the Table 1 below:

TABLE 1
diseases treatable by mixture and compositions of the invention.

| Pathogen | Strain | Gram | Disease | Abbreviation |
|---|---|---|---|---|
| Acidovorax citrulli | M6, | (−) | Bacterial fruit blotch of | Ac M6, |
| | AAC00-1 | | cucurbits | Ac AAC00-1 |
| Xanthomonas | ATCC | (−) | Black rot disease of | Xcc |
| campestris pv. | 33913 | | crucifer plants | |
| campestris | | | | |
| Xanthomonas | 97-2 | (−) | Bacterial spot disease | Xp |
| perforans | | | of tomato and pepper | |
| Pseudomonas | DC3000 | (−) | Bacterial spot disease | Pst |
| syringae pv. tomato | | | of tomato | |
| Clavibacter | NCPPB | (+) | Bacterial canker and | Cmm |
| michiganensis subsp. | 382 | | wilt of tomato | |
| michiganensis | | | | |
| Streptomyces scabies | Av | (+) | Potato common scab | Ssc |

In some embodiments, the plant disease is caused by a fungi selected from Ascomycetes and Basidiomycetes.

Mixtures of the invention are also useful against the methicillin-resistant strain of Staphylococcus aureus (MRSA).

Apart from general purpose formulations and compositions comprising mixtures of the invention, compositions of the invention may be made into pharmaceutical compositions. Such compositions comprise a mixture of the invention, a pharmaceutically acceptable carrier and optionally one or more additives or pharmaceutically active agents. The “pharmaceutically acceptable carrier” may be any vehicle which delivers the active components to the intended target and which does not cause harm to humans or other recipient organisms. Useful carriers include, for example, water, acetone, ethanol, ethylene glycol, butane-1, propylene glycol, 3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990. The pharmaceutical composition may be formulated in any form appropriate to the mode of administration, for example, solutions, colloidal dispersions, emulsions (oil-in-water or water-in-oil), suspensions, creams, lotions, gels, foams, sprays, aerosol, ointment, tablets, suppositories, and the like.

The pharmaceutical compositions can also comprise other optional materials, which may be chosen depending on the carrier and/or the intended use of the composition. Additional components include, but are not limited to, antioxidants, chelating agents, emulsion stabilizers, e.g., carbomer, preservatives, e.g., methyl paraben, fragrances, humectants, e.g., glycerin, waterproofing agents, e.g., PVP/Eicosene Copolymer, water soluble film-formers, e.g., hydroxypropyl methylcellulose, oil-soluble film formers, cationic or anionic polymers, and the like.

Peptides in mixtures of the invention may be presented in compositions of the invention in pharmaceutically acceptable salt forms. Pharmaceutically acceptable salts include acid addition salts (formed with the free amino groups of the polypeptide), which may be formed with inorganic acids, such as for example, hydrochloric or phosphoric acid, or with organic acids such as acetic, oxalic, tartaric, and the like. Suitable bases capable of forming salts with the mixtures of the present invention include, but are not limited to, inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

Mixtures of the invention may be used for hygienic purposes, as a disinfectant, for food preservation, for veterinary use, or for agricultural use.

Mixtures or compositions comprising same may be used to treat or prevent diseases. In some embodiments, the mixtures may be used in agriculture for the management of a wide variety of plant diseases. The plants may be, but not limited to, citrus, tomatoes, peppers, bananas, mangos, pears, grapes, apples and peaches.

In some embodiments, mixtures of the invention may be used for treating a plant species of a family selected from the group consisting of Cucurbitaceae, Solanaceae, and Cruciferae.

Mixtures of the invention can also be used individually or in combination with other components for disinfecting objects or agents. The term “disinfecting” or “sterilizing” encompasses preventing, inhibiting, and/or alleviating microbial growth. The amount of each component used will depend on the purpose of the use, e.g., disinfecting medical or surgical equipment, and disinfecting tissue culture equipment, incubators, hoods, dishes, and the like. The mixtures may also be used as disinfecting solutions for cleaning contact lenses, as general cleaning products and as products for enhancing the ocular comfort of patients wearing contact lenses; other types of ophthalmic compositions, such as ocular lubricating products, and the like. The concentration determined to be necessary for the such purposes can be functionally described as an amount that is effective to disinfect or sterilize a surface or an object.

In some embodiments, mixtures of the invention may be used for food preservation or for preventing microbial growth on or in a perishable product, e.g., for prolonging storage life of a food product.

In some embodiments, mixtures of the invention can be used in veterinary compositions as alternative actives to antibiotics.

Mixtures of the invention may also be used for therapeutic or cosmetic purposes. In a further aspect, the invention provides a method for disinfecting an object, the method comprising contacting an object with an effective amount of a mixture or composition of the invention.

In a further aspect, the invention provides a method for treating an infection in a subject, the method comprising administering to the subject a therapeutically effective amount of a mixture or a pharmaceutical composition comprising same.

The term “treating” or any lingual variation thereof, means remedial treatment, and encompasses reducing, suppressing, ameliorating or inhibiting the disease state or a symptom associated therewith. The term “therapeutically effective amount” refers to an amount of a mixture or a pharmaceutical composition comprising same that when administered to a subject (human or non-human) is capable of exerting antifungal and/or antibacterial activity. Assays for detecting the antifungal and/or antibacterial activity are well known in the art.

The amount of the pharmaceutical composition administered to any particular subject will depend upon a variety of factors including, but not limited to, the type, location, and extent of the microbial infection as well as the age, body weight, general health, and gender of the subject, and the route of administration. In some embodiments, the administration of the pharmaceutical composition is continued until the infection is eradicated and health has been restored to the subject. In some other embodiments, the administration of the pharmaceutical composition is continued even after the infection is eradicated for prophylactic reasons or for prevention of recurrence of a disease or disorder. In some other embodiments, the pharmaceutical composition is administered before any symptoms of a disease or disorder appear, in other words, the pharmaceutical composition may be administered to a subject to prevent a disease or disorder as described herein.

Infections that may be treated by pharmaceutical composition of the invention include, but are not limited to, topical infections caused by pathogenic organisms such as bacterial infections, particularly infections caused by bacteria resistant to antibiotics, and infections caused by pathogenic fungi. In some embodiments, the pathogens, bacteria or fungi may be any of those disclosed herein.

According to a further aspect, the present invention provides a method for treating an infection (bacterial or fungal) caused by pathogenic organisms in a subject, the method comprising administering to a subject in need thereof a therapeutically effective amount of a mixture or a pharmaceutical composition comprising same.

Mixture or pharmaceutical compositions of the invention may be administered to a subject in any route of administration including, but not limited to, topical, intralesional, intravenous, intraarterial, intramuscular, intraperitoneal, oral, ophthalmic, nasal, vaginal, and rectal. Each mode of administration represents a separate embodiment of the invention. In some embodiments, the pharmaceutical compositions of the invention are useful for topical and intralesional application.

Topical administration means administration to a particular surface area of a subject's body, to affect only that area. Thus, any and all applications in which the mixtures act locally and not through the blood circulation are also encompassed in the present invention. In some of the cases, a small amount of the actives may be found in the blood circulation. Such an amount is in any case ineffective to cause any substantial side effects or therapeutic effects.

Topical administration may be used for treating or preventing acne, fungal infections of the scalp, fungal or bacterial infections related to surgical or traumatic wounds, chronic or poorly healing skin lesions (especially in diabetes), vaginal infections (vaginitis), eye and ear infections, burn wounds, infections of mouth and throat, localized infections such as chronic pulmonary infections in cystic fibrosis, emphysema and asthma.

Topical administration may also be used on severe wounds such as burns or poorly healing wounds, e.g., foot ulcers in diabetes mellitus patients, require long-term administration of antibiotics, which lead to selection of resistant bacteria such as Streptococcus pyogenes or the methicillin-resistant Staphylococcus aureus. These can be overcome by mixtures of the invention due to their wide variety and spectrum of activity and their ability to act against non-resistant and resistant bacteria and fungi.

In the case of a systemic infection or a localized infection of a tissue or part thereof that is within a subject's body, or where treatment requires, or as per a medical practitioner's advice, the mixture of the invention may be administered in an appropriate route of administration. In some embodiments, the route of administration may be intravenous administration, intraperitoneal administration, subcutaneous administration, oral administration, sublingual administration or buccal administration.

Peptides used in mixtures of the invention can be synthesized by any peptide synthesis method known in the art. Traditionally, the most widely used synthetic method is solid-phase peptide synthesis (SPPS) which allows rapid assembly of a peptide chain through successive reactions of amino acid derivatives on a porous support. There are few types of SPPS which can be utilized for the synthesis, these include Boc/Bzl SPPS (where tert-butyloxycarbonyl protecting group is utilized), Fmoc/tBu SPPS (where fluorenylmethyloxycarbonyl protecting group is utilized) and other methods where other protecting groups, such as benzyloxy-carbonyl, may be utilized. In order to synthesize longer peptides, microwave-assisted peptide synthesis methods may be used. Further, peptides may be cyclized if needed on a solid support using ON/OFF resin cyclization methods, as known in the art.

Random peptide mixtures may be prepared according to methods detailed in (1) Z. Hayouka, S. Chakraborty, R. Liu, M. D. Boersma, B. Weisblum, S. H. Gellman, Interplay among subunit identity, subunit proportion, chain length, and stereochemistry in the activity profile of sequence-random peptide mixtures, J. Am. Chem. Soc. 135 (2013) 11748-11751; and (2) S. Topman, D Tamir-Ariel, H. Bochnic-Tamir, T. Stern Bauer, S. Shafir, S. Burdman, Z. Hayoukal, Random peptide mixtures as new crop protection, Microbial Biotechnology (2018) 11(6), 1027-1036. Each of these references is incorporated herein in full.

Solution polymerization and solid state techniques may be used for preparing the random peptide mixtures. Where one or more peptide in a mixture is in cyclic form, these cyclized peptides may be prepared according to methods known in the art, including those described above. For example, cyclization may be performed between a carboxyl and an amino termini of a peptide. Alternatively or additionally, cyclization may be performed between a functional group of an amino acid, for example an ε-amino group of Lys, and a carboxyl terminus of the peptide (Tsubery et al., (2000) J. Med. Chem. 43: 3085-3092).

The inventors have further developed a protocol for entrapping a peptide mixture or a random peptide mixture (RPMs) according to the invention, or peptides in general, in copper. Such compositions of the invention are provided as solid composite materials.

Since the results indicate that a combination of cationic lysine and hydrophobic leucine produced peptide mixtures with potent antibacterial activity against various bacteria including MRSA, this combination was elected as a model mixture for entrapment within copper. The resulting composite proved to be an efficient as a growth inhibitor of e.g., methicillin resistant Staphylococcus aureus (MRSA).

Thus, the invention further provides a composite of at least one metal (e.g., zinc, nickel, aluminum, copper, silver and others) or any alloy or an oxide form thereof entrapping a random peptide mixture (RPM).

In some embodiments, the composite is of copper metal/copper oxide and a random peptide mixture (RPM).

Composites of the invention are configured or adapted for use as crop protection agents or as preservatives or as active materials in medical devices or equipment, e.g., in bandages, patches, masks and others, and as such constructed to permit release of the at least one agent material therefrom together with copper ions. To achieve effective crop protection or preservation, composites of the invention may be in the form of a mixture of composites, wherein each composite is separately prepared to contain a separate RPM mixture and then mixed or combined to provide a composite combination of two or more composite materials. In a composite combination, each composite may be different in, inter alia, the active material it comprises, the amount of the active material, the relative amount of one composite in comparison to another in the combination, and potentially other properties that one or more of the composites may be endowed after it is prepared (endowed by any one post-treatment process).

As used herein, a “composite” is be understood to encompass a multi-component metallic-based material, comprising one or several or different phase domains, one of which being a continuous metal phase. In some embodiments, the metal phase of a composite of the invention, being also regarded as the matrix material, comprises a copper metal/copper oxide, and in some other embodiments, the metal phase comprises a metal different from copper, such as zinc, nickel, aluminum and others.

The metal/copper oxide matrix, being a continuous metal-containing phase, entraps the RPM in nanopores or cages present therein. The matrix comprises a metal such as copper metal and/or an oxide form thereof. In some embodiments, the matrix may further comprise trace amounts of a metal reducing agent, which may be any reducing agent, e.g., in the form of a metal or a metal oxide, different from copper, any non-oxide form of copper, additional oxide forms of copper, and elemental traces of sulfur containing materials.

Where the metal is copper, the oxide may be any copper oxide. The copper oxide may be selected from copper (I) oxide, Cu₂O; copper (II) oxide, CuO; copper (III) oxide, Cu₂O₃; and copper peroxide, CuO₂.

In some embodiments, the copper oxide is cuprite.

Where the matrix comprises copper metal and two or more copper oxides, the amount of each of the copper oxides may vary based on the composition and the method of its preparation. In some embodiments, the matrix consists essentially copper metal and cuprite. The term “consists essentially” is used to indicate that the matrix composition may comprise additional metals or metal oxides in trace amounts. In some embodiments, the composite comprises trace amounts of zinc (Zn), sulfur (S) and oxygen (O)-containing materials.

The RPM is entrapped, incorporated, held, contained or otherwise encompassed in nanopores or cages present along the copper metal/copper oxide matrix, in a form that retains its activity, and further in an amount and form that is sufficient to release from said nanopores or cages and exert its effect. As the RPM is in intimate interaction with the copper metal/metal oxide matrix, release of the RPM from the matrix is accompanied with concomitant release of copper ions. Without wishing to be bound by any theory or mechanism, the release of both RPM and of the copper ions endow an antimicrobial or antifungal effect that is greater than that observed for each of the components separately.

Alternatively, the composite may be regarded as a bioactive material having a bioactive surface capable of exerting its effect not only through the release of the RPM in combination with copper ions, but also through contact of the bioactive surface with a microbial population or a surface or a medium comprising or associated with a microbial population. Such microbial population may be in the form of a biofilm.

To achieve prolonged release, the RPM is configured for a timed (prolonged and/or sustained) controlled release from the copper matrix. The controlled release profile may be predetermined by changing the parameters of the composite, such as the elementary grain size, compactization under pressure, external coatings, impregnation of composite in suitable carriers, matrix pore size, and weight ratios of the composite components. In some embodiments, the release of RPM from a composite of the invention is in a rate of about half-content of the material per hour to about half-content of the material per month. In some embodiments, a composite of the invention controllably releases the entrapped material from within its metallic matrix.

The random peptide mixture used in metal composites of the invention may comprise any of the peptides disclosed herein. In some embodiments, the peptides are short peptides, wherein each of the peptides in a random mixture composite comprises or consists between 4 and 15 amino acids that are selected amongst hydrophobic and cationic amino acids. In other embodiments, the peptides are long peptides, wherein each of the peptides comprises or consists between 16 and 50 amino acids that are selected amongst hydrophobic and cationic amino acids, as defined herein. Thus, composites of the invention may comprise RPM wherein each of the peptides in the RPM comprises or consists between 4 and 50 amino acids that are selected amongst hydrophobic and cationic amino acids.

In some embodiments, the RPM comprises a mixture of peptides having between 4 and 15 amino acids, as defined hereinabove, which may be conjugated or non-conjugated to fatty acids. In some embodiments, the RPM comprises a mixture of peptides having between 20 and 50 amino acids. In some embodiments, the peptide consists 20, 25, 30, 35, 40, 45 or 50 amino acids, wherein each represents a separate embodiment of the invention. In some embodiments, the number of amino acids is 20.

The amino acids constructed the RPMs used in composites of the invention are selected as indicated hereinabove. In some embodiments, the RPM comprises peptides having a hydrophobic amino acid and a cationic amino acid in a ratio (e.g., a molar ratio) of 10:90, 70:30, 50:50, 70:30 or 90:10, wherein each possibility represents a separate embodiment of the invention.

In some embodiments, the RPM comprises peptides having a leucine:lysine or phenylalanine:lysine ratio (molar ratio) of 10:90, 70:30, 50:50, 70:30 or 90:10, wherein each possibility represents a separate embodiment of the invention.

In some embodiments the peptides of the RPM are covalently connected to a fatty acid, as defined and selected hereinabove.

Composites of the invention may be used as active agents in a variety of crop protection applications as well as preservative agents for materials prone to spoilage, such as foods and beverages. The composites are effective in the eradication, treatment or prevention of a disease in a plant or plant material that is caused by bacteria or fungi. The microbial or fungal disease may be to any plant including trees, fruit trees, bushes, vegetables, garden plants, flowing plants, in a closed growing environment, gardens, garden houses, greenhouses, citrus groves, and others. The composite may be contacted with the plant pre-harvest while in the field, greenhouse etc, or post-harvest including contact with the full pelts, fruits stems, leaves, flowers, roots or seeds of the plant.

In another aspect, the invention provides a composition, e.g., pharmaceutical composition, comprising a composite of the invention for use in the treatment or prevention of MRSA infections, for hygienic purposes or for disinfecting surfaces or objects that may be infested or associated or in contact with MRSA.

In some embodiments, the composition is for use in the treatment or prevention of MRSA infections, by utilizing e.g., topical administration.

In some other embodiments, the composition is for hygienic purposes.

In some embodiments, the composition is for disinfecting surfaces or objects that may be infested or associated or in contact with MRSA.

Composites of the invention may thus be regarded as having antimicrobial and antifungal properties. Additionally, the composites may be used as preservatives for preserving a state of a product that is prone to spoilage. Such products may be any product having a limited shelf-life that can undergo degradation or decomposition or generally can be caused to undergo spoilage by bacteria or fungi. These products may be foods, beverages, fruits and vegetables, medicinal products, veterinary products, and any other product prone to spoilage. Thus, composites of the invention may additionally be used for prolonging the shelf-life of a product.

Composites of the invention may be used as is or may be formulated in solid or liquid formulations for ease of application. Depending on the intended use, formulations of the invention may be produced in a suitable liquid or solid carrier. For agricultural applications, the carrier may be a solid or a liquid that is acceptable for spraying or for deposition in the ground or placing in the irrigation system. For pharmaceutical applications, the carrier may be a solid or a liquid selected amongst pharmaceutically acceptable carriers.

Notwithstanding any form, composites of the invention may be used in methods of eradicating, treating, suppressing or preventing bacterial or fungal infestation or in methods of preserving products prone to spoilage.

Composites of the invention may also be used in methods of treating a bacterial or fungal disease in a subject (human or animal subject) suffering therefrom.

In some embodiments, the method of treating comprising administering an effective amount of said composite to a subject suffering from a disease or disorder that can be alleviated, prevented, inhibited or treated using the composite as described herein.

In another aspect, there is provided a method of preventing, eradicating or treating a plant disease caused by bacteria or fungi, the method comprising applying an effective amount of a composite of the invention to a part of the plant. The plant part may be the bark, leaves, roots, fruit, flowers, etc, wherein the plant part is infested with the disease, suspected of having been attracted the disease, or to be prevented from attracting the disease.

The invention further provides a method of preserving at least one product prone to spoilage, the method comprising adding an effective amount of the composite, neat or in a solid or liquid formulation, to the product.

The composites may be applied to any part of the plant, by e.g., spraying, or via the roots by soil treatment. The composites may be applied in liquid or solid formulations before infection is observed, during a period of infestation or after infection has been eradicated.

The invention further provides a method of preparing a composite of the invention, the method comprises treating a copper metal precursor with at least one reducing agent, being optionally at least one metal, in the presence of RPM, under conditions permitting reduction of the copper precursor and formation of the composite.

In some embodiments, the copper precursor is at least one copper salt or at least one copper complex. In some embodiments, the copper precursor is a copper salt, optionally selected from copper sulfate, copper chloride and others.

In some embodiments, the at least one reducing agent is optionally a metal, the metal being different from copper metal. In some embodiments, the metal is Al, Fe, Zn, Li and others. In some embodiments, the metal is Zn.

In some embodiments, the reducing agent is selected from sodium amalgam, sodium alloys, diborane, borohydrides such as sodium borohydride, agents containing the Fe²⁺ ion, agents containing the Sn²⁺ ion, sulfites, dithionates, thiosulfates, iodides, formic acid, ascorbic acid, phosphites, hypophosphites, and others.

In some embodiments, reduction is achieved by electrochemical means.

Reduction of the copper precursor in the presence of RPM and formation of a composite, takes place under conditions selected from:
(1) A ratio of the copper metal precursor to the reducing agent being between 1:1 and 1:10, metal precursor:reducing agent; in some embodiments, the ratio is 1:1;
(2) An incubation at a temperature between room temperature (23-30° C.) and 50° C.; in some embodiments, the composition is incubated at room temperature;
(3) Incubation for a period of between 5 hours and 24 hours.

In some embodiments, the incubation is conducted in a room temperature, wherein in the context of the present invention, a room temperature is a temperature between 20° C. to 25° C.

In some embodiments, the incubation is conducted in a temperature of 23° C.

In some embodiments, the composite is incubated for 24 hours.

In some embodiments, the conditions of the formation of the composite further selected from mixing the solution for a period of between 5 to 24 hours.

In some embodiments, the mixing is for 24 hours.

The composite thus obtained is in the form of a copper matrix of copper metal and copper oxide, e.g., typically cuprite, entrapping, as defined, an amount of the RPM. In some embodiments, the RPM is conjugated to a fatty acid.

The inventors of the present invention have also developed additional composites which exhibit unique antimicrobial, antifungal and antibacterial properties. These composites comprise a metal entrapping an amount of an active agent being in a form of a fatty acid substituted cationic amino acid, i.e., a conjugate of a fatty acid and a cationic amino acid.

Lauroyl arginate ethyl ester (ethyl lauroyl arginate; LAE) is a modified amino-acid derived from arginine, in which the arginine amino terminus is associated with lauric acid, while the carboxy terminus is protected by an ethyl ester (Structure 1). This small molecule was selected to assess the effectiveness of the technology because of its well-characterized antimicrobial activity against a wide range of bacteria [8] and well established activity as a food preservative (commonly known as E243).

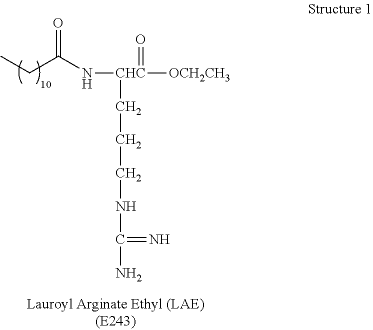

Thus, in another one of its aspects, there is provided a composite material in the form of a metal (or metal matrix) entrapping a conjugate of at least one cationic amino acid and a fatty acid. The conjugate is structured of an amino acid and a fatty moiety that may be derived from a fatty acid or from any derivative that can be used in the preparation of the conjugate.

The metal may be as defined and selected herein. In some embodiments, the metal is copper.

In another one of its aspects, the invention provides a composite material in the form of a copper metal/copper oxide matrix entrapping a conjugate of at least one cationic amino acid and a fatty acid (or entrapping a cationic amino acid that is covalently associated with a fatty moiety, as disclosed herein, that is not necessarily derived from a fatty acid).

The active agent, being a cationic amino acid, as defined and selected herein, conjugated to a fatty moiety. In some embodiments, the amino acid is constructed of a cationic amino acid such as lysine, arginine, histidine, ornithine, di-amino butyric acid (Dab) or di amino propionic acid (Dap) and a fatty moiety that is substituted on the N terminal of the amino acid.

The fatty moiety may be derived from an organic acid, as defined and selected herein, that comprises least eight carbon atoms. The fatty moiety may be derived from an acid selected from palmitic acid, decanoic acid, undecanoic acid, dodecanoic acid, myristic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid and cerebronic acid.

In some embodiments, the cationic amino acid is lysine or arginine or any derivative thereof. In some embodiments, the fatty acid is undecanoic acid or palmitic acid.

In some embodiments, the conjugate is of undecanoic acid and lysine or arginine. In some embodiments, the conjugate is of palmitic acid and lysine or arginine.

In some embodiments, the conjugate is of undecanoic acid and arginine, being referred to as lauroyl arginine or a derivative thereof (e.g., an ester such as ethyl ester thereof).

The conjugate used in a metal matrix as defined herein, e.g., lauroyl arginine, may be a compound wherein the acid side group of the cationic amino acid, e.g., arginine is free or substituted (or blocked). The substituted derivative may thus be an ester, an amide, an acid anhydride or may be in the form of a thio carboxylic acid, or in any other form.

In some embodiments, the derivative is an ester or an amide of lauroyl arginine.

In some embodiments, the ester substitution on any of the cationic amino acids discussed herein is an ester of an alcohol comprising between 1 and 10 carbon atoms. In some embodiments, the ester is of an alcohol comprising 1, 2, 3, 4, 5, 6, 7, 8, 9 or 10 carbon atoms. In some embodiments, the lauryl arginine ester is of an alcohol comprises between 1 and 5 carbon atoms. In some embodiments, the alcohol is methanol, ethanol, n-propanol, iso-propanol, n-butanol, iso-butanol and tert-butanol.

In some embodiments, the ester derivative is a methyl ester.

In some embodiments, the ester derivative is an ethyl ester.

In some embodiments, the derivative is an amide of the cationic amino acid, e.g., of lauroyl arginine. In some embodiments, the derivative is an alkyl amide or a dialkyl amide, wherein the alkyl comprises between 1 and 10 carbon atoms. Where the derivative is a dialkyl amide, each of the alkyl groups may be the same or different, each being selected from alkyls comprising between 1 and 10 carbon atoms.

In some embodiments, the amide derivative is lauroyl arginine amide.

The conjugate, e.g., lauroyl arginine or a derivative thereof is entrapped, incorporated, held, contained or otherwise encompassed in nanopores or cages present along the copper metal/copper oxide matrix, in a form that retains its activity, and further in an amount and form that is sufficient to release from said nanopores or cages and exert its effect. As the lauroyl arginine or a derivative thereof is in intimate interaction with the copper metal/metal oxide matrix, release of the lauroyl arginine or a derivative thereof from the matrix is accompanied with concomitant release of copper ions. The release of both the lauroyl arginine or a derivative thereof and of the copper ions endow an antimicrobial or antifungal effect that is greater than that observed for each of the components separately.

To achieve prolonged release, the conjugate, e.g., lauroyl arginine or a derivative thereof is configured for a timed (prolonged and/or sustained) controlled release from the copper matrix as described in above embodiments.

Composites of the invention, including those including a conjugate of an amino acid and a fatty acid, may be used as active agents in a variety of crop protection applications as well as preservative agents for materials prone to spoilage, such as foods and beverages. The composites are effective in the eradication, treatment or prevention of a disease in a plant or plant variety that is caused by bacteria or fungi. The microbial or fungal disease may be to any plant including trees, fruit trees, bushes, vegetables, garden plants, flowing plants, in a closed growing environment, gardens, garden houses, greenhouses, citrus groves, and others.

The bacteria or fungi of which the composite are active against is as described hereinabove. The bacteria of which the plant disease is caused by and may be treated and/or prevented and/or alleviated and/or eradicated by the composites of the invention are as described hereinabove.

Composites of the aspect may be used as recited herein or as preservatives for preserving a state of a product that is prone to spoilage, as described in embodiments above. Composites of the aspect may additionally be used for prolonging the shelf-life of a product as described in embodiments above.

Composites of the aspect may be formulated in any form as described in embodiments above.

Composites of the aspect may be used in methods of eradicating, treating, suppressing or preventing bacterial or fungal infestation or in methods of preserving products prone to spoilage and also may be applied to any part of the plant in any of the formulations described in embodiments hereinabove.

Also provided are methods of preventing, eradicating or treating a plant disease caused by bacteria or fungi as described in embodiments above.

The invention further provides a method of preparing a composite of the invention, the method comprises treating a metal, e.g., copper precursor with at least one reducing agent, being optionally at least one metal, in the presence of an active agent comprising cationic amino acid conjugated to a fatty acid or a derivative thereof, under conditions permitting reduction of the copper precursor and formation of the composite.

The invention further provides a method of preparing a composite of the invention, the method comprises treating a copper metal precursor with at least one reducing agent, being optionally at least one metal, in the presence of lauroyl arginine or a derivative thereof, under conditions permitting reduction of the copper precursor and formation of the composite.

The copper precursor and the reducing agent is as described in embodiments above.

Reduction of the copper precursor in the presence of a cationic amino acid conjugated to a fatty acid and formation of a composite, takes place under conditions as described in embodiments hereinabove.

In some embodiments, the cationic amino acid which is conjugated to a fatty acid is lauroyl arginine or a derivative thereof.

The composite thus obtained is in the form of a copper matrix of copper metal and copper oxide, e.g., typically cuprite, entrapping, as defined, an amount of the active agent, e.g., lauroyl arginine or a derivative thereof.

The invention further provides use of a mixture or a composite of the invention as an antibacterial or antifungal agent or composition.

The invention also provides use of a mixture or a composite of the invention for treating and/or alleviating and/or ameliorating and/or preventing a plant or human disease or disorder as described in embodiments above.

Further provided is the use of a mixture or a composite of the invention for disinfection as described in embodiments above.

Thus, the invention provides the following embodiments of the invention disclosed herein:

A composite comprising at least one metal entrapping a mixture of peptides, each peptide comparing between 3 and 50 amino acids selected from hydrophobic amino acid and/or cationic amino acid, wherein at least a portion of the peptides or each of the peptides is optionally covalently associated to a fatty moiety.

The composite, wherein each of the peptides consists of hydrophobic amino acids and/or cationic amino acids.

The composite, wherein each of the peptides in the mixture comprises a random combination of hydrophobic amino acids and cationic amino acids.

The composite, wherein the peptides comprise natural or non-natural amino acids.

The composite, wherein the peptides comprise amino acid(s) selected from β-amino acids, γ-amino acid, D-amino acid, cyclic peptides, glycosylated peptides, acetylated peptides and any combination thereof.

The composite, wherein each of the peptides comprising between 3 and 30 amino acids.

The composite, wherein the number of amino acids is between 4 and 15.

The composite, wherein the number of amino acids is between 20 and 50 amino acids.

The composite, wherein the number of amino acids is between 5 and 20 amino acids.

The composite, wherein the number of cationic amino acids in the mixture is larger then the number of hydrophobic amino acids.

The composite, wherein the ratio of cationic amino acids to hydrophobic amino acids in the mixture is at least 1:1, at least 2:1, at least 3:1, at least 4:1, at least 5:1, at least 6:1, at least 7:1, at least 8:1 or at least 9:1.

The composite, wherein the ratio is between 1:1 and 10:1.

The composite, wherein the ratio between the hydrophobic amino acids to cationic amino acids is between 3:1 and 1:3.

The composite, wherein the cationic amino acid is selected from lysine, arginine, histidine, ornithine, di-amino butyric acid (Dab) and di amino propionic acid (Dap).

The composite, wherein the cationic amino acid is lysine or arginine.

The composite, wherein the hydrophobic amino acid is selected from proline, methionine, tryptophan, phenylalanine, leucine, isoleucine, glycine, alanine and valine.

The composite, wherein the hydrophobic amino acid is phenylalanine.

The composite, wherein the peptides comprise or consist phenylalanine and lysine.

The composite, wherein one or more peptides in the mixture is conjugated to a fatty moiety.

The composite, wherein one or more peptides in the mixture is conjugated to a fatty moiety, the fatty moiety being conjugated to the peptide at a position that is further apart from the at least one hydrophobic amino acid in the peptide.

The composite, wherein the fatty moiety is derived from saturated, unsaturated, monounsaturated and polyunsaturated fatty acids.

The composite, wherein the fatty moiety comprises at least eight carbon atoms.

The composite, wherein the fatty moiety is derived from a fatty acid selected from decanoic acid, undecanoic acid, dodecanoic acid (lauric acid), myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

The composite, wherein the fatty moiety is conjugated to the peptides N-terminals, the C-terminals, or to any functional group along the peptide chain.

The composite, wherein the peptide comprises lysine and the fatty moiety is conjugated to the lysine ε-amino group.

The composite, wherein the fatty moiety is derived from palmitic acid or lauryl acid.

The composite, comprising a combination of peptides wherein two or more of the peptides having an identical number of amino acids, but of different sequences.

The composite, being in a form of a solid mixture, a dispersion or a solution.

The composite, being antimicrobial.

The composite, having an antibacterial activity and/or an antifungal activity.

The composite, for use in the preparation of an antimicrobial composition.

The composite, wherein the composition is a pharmaceutical composition or an agricultural formulation.

The composite, wherein the mixture of peptides is a random antimicrobial peptide mixture (RPM).

The composite, wherein the at least one metal is selected from zinc, nickel, aluminum, copper and silver or any metal alloy or metal oxide thereof.

The composite, wherein the at least one metal is copper.

The composite, being in a form of copper metal/copper oxide matrix entrapping a random antimicrobial peptide mixture (RPM).

The composite, wherein the mixture comprises a plurality of conjugates, each conjugate comprising a fatty acid coupled to a peptide having between 3 and 50 amino acids, wherein the conjugate consist hydrophobic and/or cationic amino acids, and wherein the ratio in the mixture of the total hydrophobic to cationic amino acids is between 3:1 and 1:3.

The composite, wherein the ratio in the mixture is between 2:1 and 1:2.

The composite, wherein the ratio is about 1:1.

The composite, further comprising trace amounts of any one or more of a copper cation and/or a reducing agent.

The composite, capable of releasing the peptides and copper ions.

A composition comprising a composite according to the invention.

The composition, being an antimicrobial composition.

The composition, being effective against a microorganism selected from a bacterium and a fungus.

The composition, wherein the microorganism is a antibiotic-resistant bacterium.

The composition, wherein the bacterium is selected from Gram-negative and Gram-positive bacteria.

The composition, wherein the bacterium is selected from Bacillus anthracis; Clostridium botulinum; Francisella tularensis; Yersinia pestis; Burkholderia pseudomallei; Burkholderia mallei; Clostridium perfringens; Coxiella burnetii; Brucella melitensis, abortus, suis and canis; Staphylococcus aureus; Rickettsia prowazekii; Chlamydia psittaci; Food and Waterborne Pathogens such as Escherichia coli, Vibrio cholerae, Salmonella species, Shigella species, Listeria monocytogenes, Campylobacter jejuni, Yersinia enterocolitica; Mycobacterium tuberculosis; and other Rickettsia.

The composition, wherein the microorganism is methicillin-resistant Staphylococcus aureus (MRSA), vancomycin-resistant enterococci (VRE), Clostridium difficile, Acinetobacter baumannii and multi-drug resistant (MDR) Acinetobacter sp.

The composition, wherein the microorganism is selected from Xanthomonas, Agrobacterium, Erwinia, Leifsonia, Pectobacterium, Pseudomonas, Ralstonia, and Xylella. In some embodiments, the bacterium is selected from Xanthomonas campestris pv. campestris (Xcc) and Xanthomonas campestris pv. vesicatora (Xcv).

The composition, wherein the fungus is selected from Candida, Aspergillus, Cryptococcus, Histoplasma, Pneumocystis and Stachybotrys.

The composition, wherein the fungus is selected from Candida albicans, Candida amphixiae, Candida antarctica, Candida argentea, Candida ascalaphidarum, Candida atlantica, Candida atmosphaerica, Candida auris, Candida blankie, Candida blattae, Candida bracarensis, Candida bromeliacearum, Candida carpophila, Candida carvajalis, Candida cerambycidarum, Candida chauliodes, Candida corydalis, Candida dosseyi, Candida dubliniensis, Candida ergatensis, Candida fermentati, Candida fructus, Candida glabrata, Candida guilliermondii, Candida haemulonii, Candida humilis, Candida insectamens, Candida insectorum, Candida intermedia, Candida jeffresii, Candida kefyr, Candida keroseneae, Candida krusei, Candida lusitaniae, Candida lyxosophila, Candida maltose, Candida marina, Candida membranifaciens, Candida mogii, Candida oleophila, Candida oregonensis, Candida parapsilosis, Candida quercitrusa, Candida rhizophoriensis, Candida rugosa, Candida sake, Candida sharkiensis, Aspergillus fumigatus, Aspergillus flavus, Cryptococcus neoformans, Cryptococcus laurentii, Cryptococcus albidus, Cryptococcus gattii, Histoplasma capsulatum, Pneumocystis jirovecii (or Pneumocystis carinii) and Stachybotrys chartarum.

The composition, wherein the fungus is Candida albicans.

The composition, wherein the microorganism is plant related.

The composition, wherein the microorganism is a bacterium selected from Gram-negative and Gram-positive plant-pathogenic bacteria.

The composition, wherein the microorganism is a plant bacterium of a genera selected from Erwinia, Pectobacterium, Pantoea, Agrobacterium, Pseudomonas, Ralstonia, Burkholderia, Acidovorax, Xanthomonas, Clavibacter, Streptomyces, Xylella, Spiroplasma, and Phytoplasma.

The composition, wherein the bacterium is selected from Burkholderia, and Pseudomonas syringae.

The composition, wherein the bacterium is selected from Xanthomonas spp. such as Xanthomonas campestris pv. campestris (Xcc) and Xanthomonas campestris pv. vesicatora (Xcv).

The composition, wherein the bacterium is selected from Pseudomonas spp.

The composition, for inhibiting growth of strains of Clavibacter michiganensis subsp. michiganensis, Xanthomonas perforans, Xanthomonas campestris pv. campestris and Acidovorax citrulli.

The composition, wherein the microorganism is a plant fungus selected from Ascomycetes and Basidiomycetes.

The composition, for use against a methicillin-resistant strain of Staphylococcus aureus (MRSA).

The composition, for use as a hygienic composition, a disinfectant, a food preservative, a veterinary composition, or as an agricultural composition.

The composition, for use in a method of treating or preventing a disease.

The composition, for managing a plant disease.

The composition, wherein the plant is selected from citrus, tomatoes, peppers, bananas, mangos, pears, grapes, apples and peaches.

A method for disinfecting an object, the method comprising contacting the object with an effective amount of a composite of the invention or a composition comprising same.

A method for treating an infection in a subject, the method comprising administering to the subject a therapeutically effective amount of a composite according the invention or a pharmaceutical composition comprising same.

A method for treating an infection (bacterial or fungal) caused by pathogenic organisms in a subject, the method comprising administering to a subject in need thereof a therapeutically effective amount of a composite according to the invention or a pharmaceutical composition comprising same.

The method, wherein administering the composite of a composition comprising same to the subject is by topical, intravenous, intraarterial, intramuscular, intraperitoneal, oral, ophthalmic, nasal, or intralesional administration.

A disinfecting composition comprising a composite according to the invention.

A food preservative composition comprising a composite according to the invention.

A method for disinfecting an object comprising contacting the object with a disinfecting composition according to the invention.

The method, wherein the object is selected from tissue culture equipment, tissue culture media, tissue culture incubators, tissue culture hoods, and tissue culture dishes.

The method, wherein the object is selected from medical and surgical equipment.

A composite of copper or any alloy or an oxide form thereof entrapping a random peptide mixture (RPM).

The composite, wherein the metal is copper and the copper oxide is selected from copper (I) oxide, Cu₂O; copper (II) oxide, CuO; copper (III) oxide, Cu₂O₃; and copper peroxide, CuO₂.

The composite, wherein the copper oxide is cuprite.

The composite, wherein the metal consists essentially copper metal and cuprite.

The composite, wherein the RPM is entrapped in nanopores or cages present along a metal or oxide matrix.

The composite, wherein the peptides in the RPM comprise or consist between 3 and 50 amino acids selected amongst hydrophobic and cationic amino acids.

The composite, wherein the peptides in the RPM comprise or consist between 16 and 50 amino acids selected amongst hydrophobic and cationic amino acids.

The composite, wherein the peptides are conjugated to fatty moieties.

The composite, wherein the ratio of hydrophobic amino acids and cationic amino acids is 10:90, 70:30, 50:50, 70:30 or 90:10.

The composite, wherein the RPM comprises peptides having a leucine:lysine or phenylalanine:lysine ratio (molar ratio) of 10:90, 70:30, 50:50, 70:30 or 90:10.

The composite, for use in crop protection.

The composite, for use in treatment or prevention of MRSA infections, for hygienic purposes or for disinfecting surfaces or objects.

A bioactive material in the form of a composite according to the invention.

The bioactive material, for use as a crop protection agent.

The bioactive material, for use in treating or preventing a plant disease caused by bacteria.

A method of preventing, eradicating or treating a plant disease caused by bacteria or fungi, the method comprising applying an effective amount of a composite according to the invention to a part of the plant.

A method of preserving at least one product prone to spoilage, the method comprising adding to the product an effective amount of a composite according to the invention, neat or in a solid or liquid formulation.

A method of preparing a composite according to the invention, the method comprising treating a metal precursor with at least one reducing agent, being optionally at least one metal, in the presence of the peptide mixture, under conditions permitting reduction of the metal precursor and formation of the composite.

The method, wherein the metal is copper and the at least one metal is different from copper.

The method, wherein the metal precursor is at least one metal salt or at least one metal complex.

The method, wherein the metal is copper and the copper precursor is a copper salt, optionally selected from copper sulfate, copper chloride and others.

The method, wherein the reducing agent is selected from sodium amalgam, sodium alloys, diborane, borohydrides, agents containing Fe²⁺ ion, agents containing Sn²⁺ ion, sulfites, dithionates, thiosulfates, iodides, formic acid, ascorbic acid, phosphites, and hypophosphites.

The method, wherein reduction is achieved by electrochemical means.

The method, wherein the metal is copper and the conditions are selected from:
(1) A ratio of the copper metal precursor to the reducing agent being between 1:1 and 1:10, metal precursor:reducing agent;
(2) An incubation at a temperature between room temperature (23-30° C.) and 50° C.; and
(3) Incubation for a period of between 5 hours and 24 hours.

A composite material in the form of a metal (or metal matrix) entrapping a conjugate of at least one cationic amino acid and a fatty acid.

The composite, wherein the metal is copper.

The composite, wherein the fatty acid is selected from palmitic acid, decanoic acid, undecanoic acid, dodecanoic acid, myristic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid and cerebronic acid.

The composite, wherein the cationic amino acid is lysine or arginine or a derivative thereof.

The composite, wherein the fatty acid is undecanoic acid or palmitic acid.

The composite, wherein the conjugate is of undecanoic acid and lysine or arginine.

The composite, wherein the conjugate is of palmitic acid and lysine or arginine.

The composite, wherein the conjugate is of undecanoic acid and arginine.

The composite, wherein the conjugate is in a form of an ester, an amide, an acid anhydride or a thio carboxylic acid.

The composite, wherein the conjugate is an ester or an amide of lauroyl arginine.

The composite, wherein the ester is of an alcohol comprising between 1 and 5 carbon atoms.

The composite, wherein the ester is a methyl ester.

The composite, wherein the conjugate is lauroyl arginine methyl ester.

The composite, in the form of a copper metal (or metal matrix) entrapping lauroyl arginine methyl ester.

The composite, for use as an antibacterial or antifungal agent.

A mixture of a plurality of conjugates, each conjugate comprising a fatty acid coupled to a peptide of 3 to 50 amino acid residues in length, wherein the conjugate peptides consist of hydrophobic and/or cationic amino acids, and wherein the ratio in the mixture of the total hydrophobic and cationic amino acids is optionally between 3:1 and 1:3.

The mixture having at least one activity selected from the group consisting of antibacterial and antifungal activity.

The mixture, wherein the ratio is about 1:1.

The mixture, wherein the fatty acid is selected from saturated, unsaturated, monounsaturated and polyunsaturated fatty acids.

The mixture, wherein the fatty acid consists of at least eight carbon atoms.

The mixture, wherein the fatty acid is selected from palmitic acid, decanoic acid, undecanoic acid, dodecanoic acid, myristic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

The mixture, wherein the fatty acid is palmitic acid.

The mixture, wherein the hydrophobic amino acid is selected from the group consisting of phenylalanine, leucine, valine, alanine, isoleucine, and glycine.

The mixture, wherein the hydrophobic amino acid is phenylalanine.

The mixture, wherein the cationic amino acid is selected from the group consisting of lysine, arginine, histidine, ornithine, and di-amino butyric acid (Dab).

The mixture, wherein the hydrophobic amino acid is phenylalanine and the cationic amino acid is lysine.

The mixture, wherein at least one peptide is in a cyclic form.

The mixture, wherein the peptide consists of 5 amino acid residues.

The mixture, wherein the antimicrobial activity of the mixture of conjugates being higher than the activity of the same mixture in unconjugated form.

The mixture, wherein the antimicrobial activity of the mixture of conjugates is at least 10 times higher than the activity of the same mixture in unconjugated form.

A method of treating and/or preventing a plant disease, comprising administering to the plant an effective amount of a mixture according to claim the invention (short or long peptides).

The method, wherein the plant disease is caused by bacteria or fungi.
The method, wherein the plant disease is caused by antibiotic-resistant bacteria. The method, wherein the bacteria are selected from the group consisting of Xanthomonas campestris pv. campestris (Xcc) and Xanthomonas campestris pv. vesicatora (Xcv).

A pharmaceutical composition comprising the mixture of the invention.

A method for treating an infection in a subject comprising administering to a subject in need thereof a therapeutically effective amount of the pharmaceutical composition of the invention.

The method, wherein administering the pharmaceutical composition to the subject is selected from topical, intravenous, intraarterial, intramuscular, intraperitoneal, oral, ophthalmic, nasal, and intralesional administration.

The method, wherein the infection is caused by pathogenic organisms.

The method, wherein the pathogenic organisms are bacteria.

The method, wherein the bacterial infection is caused by antibiotic-resistant bacteria.

The method, wherein the infection is a fungal infection.

A disinfecting composition comprising a mixture according to the invention.

A food preservative composition comprising a mixture according to the invention.

A method for disinfecting an object comprising contacting the object with a disinfecting composition, the composition comprising as an active ingredient the mixture of the invention.

The method, wherein the object is selected from tissue culture equipment, tissue culture media, tissue culture incubators, tissue culture hoods, and tissue culture dishes.

The method, wherein the object is selected from medical and surgical equipment.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-D show the antimicrobial effects of lipo-RPMs. FIG. 1A describes minimal inhibitory concentration (MIC) of lipo-RPMs/RPMs towards Xcc after 24 h. Values ˜200 μg/ml mean that no effect was detected. FIG. 1B describes bactericidal effect over time: log reduction of Xcc (at an initial concentration of 108 CFU/ml) after exposure of 2, 10, 60 and 120 min to 25 μg/ml lipo-RPMs/RPMs. FIG. 1C describes MIC values of RPMs/lipo-RPMs towards different bacterial strains (see Table 2 for strain details) after 24 h. FIG. 1D presents the effect of p-FdK5 on bacterial spot disease of tomato. Tomato leaves were pretreated with p-FdK5 (200 μg/ml), Kocide®2000 (2,500 μg/ml) or sterilized DDW (control). After 24 h leaflets were sprayed with X. perforans 97-2 at 108 CFU/ml. Disease severity was estimated at different time points using a 0 to 6 scale. Data represent averages and standard errors (SE) of 3 experiments with 5 replicates per treatment.

FIGS. 2A-B provide assessment of cytotoxicity of lipo-RPMs in HEK293T-2 MTT assays. FIG. 2A presents percentage of viable cells in response to different concentrations of the lipo-RPM p-FdK5 and p-FdK5 specific lipopeptide mixture (SLM, a mixture of equal amounts of the 32 purified lipopeptides that compose p-FdK5) in comparison with the RPMs FdK5 and FdK20. RPMs were synthesized with a 1:1 ratio of ^{L}F and ^{D}K. FIG. 2B presents Percentage of viable cells in response to p-FdK5 lipo-RPMs that were synthesized using different ratios of ^{L}F and ^{D}K. The cytotoxic effects were determined on exponentially growing HEK293T mammalian cells using the MTT assay. After 24 h incubation, cells were stained, and the percentage of viable cells was determined using a plate reader. Data represent averages and standard errors of at least 3 independent experiments with four replicates for each lipo-RPM/RPM concentration.

FIG. 3 shows bacteriostatic effects of the 32 sequence-specific lipopeptides that compose the p-FdK5 lipo-RPM. The 32 individual lipopeptides that compose the p-FdK5 lipo-RPM were synthesized and assessed in MIC assays towards Xcc. Values ˜200 μg/ml mean that no effect was detected. Data represent averages and SE of at least 3 independent experiments with 3 replicates per lipopeptide in each experiment.

FIG. 4 shows the correlation between bacteriostatic ability and level of hydrophobicity of lipopeptides. The 32 different lipopeptides that compose the p-FdK5 mixture were synthesized individually, purified using RP-HPLC (acetonitrile and 0.1% trifluoroacetic acid in DDW were used as solvents), and tested in MIC assays with Xcc for (24 h, 28°). Each number represents a specific lipopeptide (see Table 3 for details). Spearman Rho correlation coefficient=0.8704, p<0.001

FIG. 5 presents the bacteriostatic effects of lipo-RPMs towards Xcc as determined by MIC values. p-FdK5 is the original, randomly synthesized mix. p-FdK5 SLM is a mixture of the 32 individually synthesized and purified lipopeptides that compose p-FdK5 at equal amounts. #1-15 is a mix of peptides #1 to #15, that possesses the lowest MIC values (<15 μg/ml), at equal amounts. MIC values were determined after 24 h incubation at 28° C. Data represent averages and standard errors of at least 3 independent experiments with three replicates for each compound.

FIGS. 6A-B show a comparison between different lipopeptides mixtures and individual lipopeptides in terms of bactericidal activity towards Xcc. FIG. 6A presents a comparison between different p-FdK5 SLMs (each one containing a mixture of all possible lipopeptides within each F:dK ratio group) and individual lipopeptides from each group. * and different letters indicate significant differences between treatments (p<0.05). FIG. 6B presents a comparison among 5-mer lipo-RPMs that were synthesized using different relative concentrations of F and dK. Bactericidal activity was measured after 10 min of incubation of Xcc cells (initial concentration of 10⁸ CFU/ml) with lipo-RPMs [25 μg/ml]. * indicates significant differences (p<0.05) as compared with original p-FdK5 (synthesized with equal amounts of F and dK).

FIGS. 7A-B describe antimicrobial activity towards Xcc of RPMs and SLMs with different F:dK ratios. FIG. 7A shows bacteriostatic effect (MIC values) was determined after 24 h incubation at 28° C. The highest concentration tested was 200 μg/ml, therefore values ˜200 μg/ml mean that no effect was detected under tested conditions. Data represent averages and standard errors of at least 3 independent experiments with three replicates per treatment. FIG. 7B. shows the bactericidal effect was measured after 10 min incubation at 28° C. of 10⁸ CFU/ml Xcc cells with the different lipopeptides [25 μg/ml]. Asterisks indicate significant differences between groups (p<0.05). Data represent averages and standard errors of at least 3 independent experiments with two replicates per treatment in each experiment.

FIG. 8 is schematic representation of the checkerboard assay plate used to assess synergistic effect. Assays were conducted in 96-well containing two-fold dilutions of the two tested lipopeptides combined in different concentrations starting from 4×MIC values (columns 1-6). For each tested lipopeptide, two columns containing two-fold serial dilutions of the tested lipopeptide alone, starting at concentrations of 4×MIC values were used to evaluate the MIC (columns 7-10). The remaining columns were used as positive control (wells containing Xcc bacteria at a concentration of ˜10⁸ CFU/ml without tested lipopeptides) and negative control (wells containing NB without bacteria, columns 11 and 12, respectively).

FIG. 9 shows the entrapment of leucine-lysine random peptide mixtures (LK) in a copper matrix. Copper ions were reduced by metallic zinc in the presence of leucine-lysine random peptide mixtures to form tight aggregated copper nanocrystals with the peptides chains trapped inside. Copper oxide forms upon exposure of the process to air, and becomes part of the final matrix.

FIGS. 10A-B describe characterizing the new composites material. FIG. 10A shows energy dispersive X-ray analysis of LK20-mer@[Cu]. FIG. 10B shows structure and morphology of [Cu] composites (left column, LK20-mer@[Cu] (right column) and LK10-mer (middle column) composites.

FIGS. 11A-B show chemical analysis of LK entrapment. FIG. 11A shows organic elemental analysis of the designed composites presented as percentage of weight. The chart shows the average and standard errors of 3 independents repeats. FIG. 11B shows thermal gravity analysis of composites. [Cu]—smooth line, LK10-mer@[Cu]—dashed line, LK20-mer@[Cu]—dotted line.

FIGS. 12A-B show Methicillin-resistant Staphylococcus aureus growth inhibition in the presence of 200 ppm [Cu] (squares), LK20-mer@[Cu] (circles), LK10-mer@[Cu] (triangles), 4 μg/mL free LK 20-mer (asterisks) and without addition (control, black circles). FIG. 12A demonstrates the growth curves from one representative experiment. FIG. 12B shows the calculated average and standard errors of 7 independent repeats of growth inhibition after 8 and 24 h.

FIGS. 13A-B demonstrate the effect of physical mixing of copper sulfate (CuSO₄) ions and free LK RPM. Determining the antimicrobial activity by the inhibition of Methicillin-resistant Staphylococcus aureus growth exhibited by physical mixing of copper sulfate (CuSO₄) ions and free LK RPM. The results are the average and standard error of 3 independent repeats. FIG. 13A demonstrates the results where LK20-mer was mixed. FIG. 13B demonstrates the results where LK10-mer was mixed.

FIGS. 14A-C present TGA results of three composites. FIG. 14A shows TGA results for LAE×1.0@Cu. FIG. 14B shows TGA results for LAE×0.5@Cu. FIG. 14C shows TGA results for LAE×0.25@Cu.

FIG. 15 demonstrated Elemental analysis. This analysis allows quantification of the elements carbon (C), nitrogen (N), hydrogen (F) and sulfur (S). Since the first three elements are present in LAE, this test serves as an additional analysis to quantify the amount of LAE entrapped within the Cu matrix.

FIGS. 16A-B show EDS results for composites. FIG. 16A shows results for LAE×1.0@Cu. FIG. 16B shows results for @Cu.

FIGS. 17A-B show representative images of the composites. FIG. 17A shows images of LAE×1.0@Cu composite. FIG. 17B shows images of @Cu sample.

FIG. 18 presents the bactericidal activity of the three generated composites carrying different doping amounts of LAE-LAE×1.0@Cu, LAE×0.5@Cu and LAE×0.25@Cu- at concentration of 25 ppm. Controls included @Cu (composite-like compound without LAE) and a commercial bactericide, Kocide (DuPont), both at 25 ppm. An additional control was LAE at 1.25 ppm, which is the expected concentration of this molecule in 25 ppm of the LAE×1.0@Cu composite.

FIG. 19 shows an experiment where A. citrulli M6 cells were treated with two concentrations, 12.5 and 25 ppm, of the LAE×1.0@Cu composite. Controls were @Cu and LAE at the concentrations that corresponded to their expected concentrations in the composite treatments (12.5 ppm @Cu and 0.625 ppm LAE for the 12.5 ppm LAE×0.5@Cu treatment, and 25 ppm @Cu and 1.25 ppm LAE for the 25 ppm LAE×1.0@Cu treatment.

FIG. 20 shows the bactericidal activity of different concentrations of the LAE×1.0@Cu composite (35 and 55 ppm) with Kocide at 55 ppm was compared. These experiments were carried out using a different plant-pathogenic bacterium, Xanthomonas perforans 97-2.

FIGS. 21A-C show the effect of LAE@[Cu] on disease severity of melon leaves inoculated with A. citrulli M6. A. Representative leaves of the different disease severity scores. B. Average disease severity scores of the different treatments. C. Effects of the different treatments on disease severity as expressed in percentage of disease inhibition. Results represent averages and standard errors from 3 independent experiments, in which each treatment contained 20 to 40 leaves (replicates). The data were statistically analysed by one-way analysis of variance (ANOVA) and Tukey's honest significant difference (HSD) test (p<0.05).

FIG. 22 presents thermal gravimetric analysis (TGA) results of three composites with different doping concentrations of LAE. LAE×1.0@[Cu], 4.117 mM (1733 ppm); LAE×0.5@[Cu], 2.058 mM (866 ppm); LAE×0.25@[Cu], 1.029 mM (433 ppm).

FIG. 23 shows percentage of the organic elements in three different doping concentrations of LAE: LAE×1.0@[Cu], LAE×0.5@[Cu], LAE×0.25@[Cu] and @[Cu] were examined by elemental analyses. These results are averages and standard deviations of at least two repeats per sample.

FIGS. 24A-B show energy dispersive spectroscopy (EDS) results. FIG. 24A. LAE×1.0@[Cu]. FIG. 24B. @[Cu]. Each spectrum represents the results of one analysis out of five independent analysis (with different batches) with similar results.

FIGS. 25A-B show results of X-ray diffraction (XRD). FIG. 25A. LAE×1.0@[Cu]. FIG. 25B. @[Cu]. Each spectrum represents the results of one analysis out of three independent analysis (with different batches) with similar results.

FIGS. 26A-B show LAE release profile from LAEX1.0@[Cu]. FIG. 26A shows standard curve of LAE, different concentrations of LAE in DDW (0.5, 1, 2, 3, 4, 5 & 10 ppm) at 30° C., shaking conditions. FIG. 26B shows LAE release profile from 400 ppm LAEX1.0@[Cu] (20 mg in 50 ml DDW), at 30° C., shaking conditions for different time intervals each (t=0, 1.5, 3, 6, 9 and 24 h) (Mean±SE, n=3).

FIG. 27 shows Cu⁺2 release profile from LAEX1.0@[Cu] and @[Cu].

FIGS. 28A-B demonstrate MALDI TOF MS representative spectra of leucine (L) lysine (K) random peptide mixtures after completing the synthesis. FIG. 28A shows the results of LK 20-mer peptide. FIG. 28B shows the results of LK 10-mer peptide.

FIGS. 29A-D present EDAX analysis of copper reduced by zinc at a ratio of 1:1 or 1:1.2 and with or without a 4N HCl acid wash. FIG. 29A shows the results for Cu:Zn 1:1.2. FIG. 29B shows the results of Cu:Zn 1:1.2 w/ acid wash. FIG. 29C shows the results of Cu:Zn 1:1. FIG. 29D shows the results of Cu:Zn 1:1 w/ HCL 4N wash.

FIGS. 30A-C depict the X-ray diffraction (XRD) results of composites. FIG. 30A shows the results for Cu—Zn. FIG. 30B shows the results for Lk10@Cu. FIG. 30C shows the results for Lk20@Cu.

FIGS. 31A-B show the thermal gravity analysis of composites and first derivative. In the utilized assay, the amount of the organic compound entrapped in the copper matrix is quantified.

### DETAILED DESCRIPTION OF EMBODIMENTS

### Materials and Methods Regarding RPM and Fatty Acid Conjugates

Chemicals (i): Fmoc-protected α-amino acids with acid-labile side-chain protecting groups were purchased from Novabiochem. N-hydroxybenzotriazole (HOBt), N,Ndimethylformamide (DMF), and N,N-diisopropylethylamine (DIEA) were purchased from Sigma-Aldrich. 2-(1H-Benzotriazole-1-yl)-1,1,3,3-tetramethyluronium hexafluoro phosphate (HBTU) was purchased from Anaspec. Rink Amide resin was purchased from Novabiochem. Dehydrated LB culture medium (244610) was obtained from BD (Franklin Lakes, N.J.). All other chemicals were purchased from Sigma Aldrich and used without purification.

Synthesis of sequence-random peptide mixtures (ii): Random peptide mixtures were synthesized using microwave irradiation on Rink Amide resin (Substitution 0.2 mmol/gr, 25 μmol) in Alltech filter tubes. Coupling reactions were conducted with binary combinations of protected amino acids, with a freshly prepared stock solution that contained the protected amino acids in defined proportions, which was used for each coupling step. For example, the synthesis of a mixture of 5-mer mixture began with the preparation of a stock solution containing a total of 20 equiv. of Fmoc-protected amino acids in the appropriate molar ratio. Before each coupling step, an aliquot containing 4 equiv. (100 μmol) of the amino acid mixture was activated with 4 equiv. of HBTU, 4 equiv. of HOBt, and 8 equiv. of DIEA, in DMF. The use of a single stock solution was intended to reduce errors associated with weighing out protected amino acids. For the synthesis of a mixture of L-Leu+L-Lys 5-mers with a 1:1 molar ratio of amino acids, the stock solution was prepared by dissolving 10 equiv. of Fmoc-L-Leu-OH and 10 equiv. of Fmoc-L-Lys(Boc)-OH in 5 mL DMF. For each coupling reaction, 1 mL (which contains a total of 4 equiv. of protected amino acids, in this case) from the amino acids mixture stock solution was activated with 4 equiv. of HBTU, 4 equiv. of HOBt, and 8 equiv. of DIEA. After the activated amino acid solution was added to the solid-phase synthesis resin, the reaction mixture was heated to 70° C. in a MARS V multimode microwave (2 minute ramp to 70° C., 4 minute hold 70° C.) with stirring. Fmoc deprotection reactions used 20% piperidine in DMF. Reaction solutions were heated to 80° C. in the microwave (2 minute ramp to 80° C., 2 minute hold 80° C.) with stirring. After each coupling/deprotection cycle the resin was washed 3 times with DMF. Upon completion of the synthesis, the peptide mixture was cleaved from the resin by stirring the resin in a solution containing 95% trifluoroacetic acid (TFA), 2.5% water, and 2.5% triisopropylsilane for 3 hours. The peptide mixture was precipitated from the TFA solution by addition of cold ether. The precipitated peptide mixture was collected by centrifugation. Ether was removed, and the pellet was dried under a stream of nitrogen, frozen in dry ice and lyophilized.

For a fatty acid attachment, after the last step of Fmoc deprotection, palmitic acid or other fatty acids was attached to the N-terminus of the peptide by acylation. 4 equiv. of fatty acid was dissolved in 1 mL DFM 4 equiv HBTU 4 was added and mixed with 8 equiv. of DIEA and was shaken over night.

Antibacterial assays (iii): The bacterial strains used in these assays were Xanthomonas campestris pv. campestris (Xcc) and X. campestris pv. vesicatoria (Xcv). The antibacterial activity for random lipo-peptide mixtures was determined in sterile 96-well plates (BD Falcon 353072 tissue culture plates) by a broth microdilution method. Bacterial cells were grown overnight at 37° C. in agar, after which a bacterial suspension of approximately 2×10⁶ CFU/mL in LB growth medium was prepared. Aliquots (50 μL) of this suspension were added to 50 μL of medium containing the peptide mixture in 2-fold serial dilutions for a total volume of 100 μL in each well. The plates were then incubated at 37° C. for 6 h. Bacterial growth was determined by measuring the optical density (OD) at 650 nm using a Molecular Devices Emax precision microplate reader. The lowest concentration of peptide mixture that causes complete inhibition of bacterial growth is defined as the minimum inhibitory concentration (MIC). MIC values were reproducible to within a factor of 2 and are reported as the median of at least two individually determined values. For all assays, Magainin 2 amide was tested in parallel as control.

Synthesis of random-sequence cyclic peptides conjugated to fatty acids (iiii): The cyclic peptides are synthesized by a solid-phase method as described in section (ii) above. To keep the fatty acid on the N-terminus of the peptides the cyclization step is performed using side chains of different amino acids (e.g., Glu, Asp, Lys, Orn, and Dab) along the peptides to have a conformational library while still bound to the resin. The cyclic peptides are further characterized by MALDI TOF mass spectrometer and subjected to amino acid analysis to confirm their hydrophobic:cationic amino acids ratio. After the last step of cyclyzation, palmitic acid or another fatty acid is attached to the N-terminus of the peptide by acylation as was described before.

### Example 1

Synthesis of 5-mer random peptide mixtures conjugated to fatty acid: The following mixtures were synthesized as described in the materials and methods section (ii). Palmitic acid (PA) was conjugated to 5-mer or 10-mer random C-amidated diastereomeric peptides to yield the mixtures conjugates. The peptides contained the hydrophobic amino acid Phe and the positively charged amino acid D/L-Lys (^{L}F^{L}K or FK and ^{L}F^{D}K or FDK).

### Example 2

Antibacterial activity of the random peptide mixtures conjugated to fatty acid: The antibacterial activity of the mixtures was examined as described in the materials and methods section above. The antibacterial activity of random 5-mer (FDK 5-mer) and 10-mer (FDK 10-mer) random mixtures was compared to same mixtures comprising 5-mer, 10-mer and 20-mer random sequence peptides without the palmitic acid (PA). As shown in Structure 1 above, 5-mer mixtures not coupled to PA had no activity, while 5-mer random peptides coupled to PA exhibited high activity (MIC of 12 μg/ml). The antibacterial activity of 5-mer FDK coupled to PA was 2 times higher than the 20-mer random peptide mixture without PA. The 10-mer FDK, while not showing antibacterial activity without PA, exhibited similar activity (MIC of 25 μg/ml) to the 20-mer control when coupled to PA.

### Properties and Characteristics of RPMs and its Relation to Anti-Bacterial Activity

The effects of the attachment of a fatty acid residue to RPMs on their antimicrobial activity was explored in the herein example. Xanthomonas campestris pv. campestris (Xcc) ATCC 33913 was used as a model plant-pathogenic bacterium. Xcc causes black rot disease, which is considered as the most important disease of brassica crops worldwide. Recently, it was shown that both FK20 and FdK20 RPMs are highly active towards this bacterium in in vitro and in planta assays.

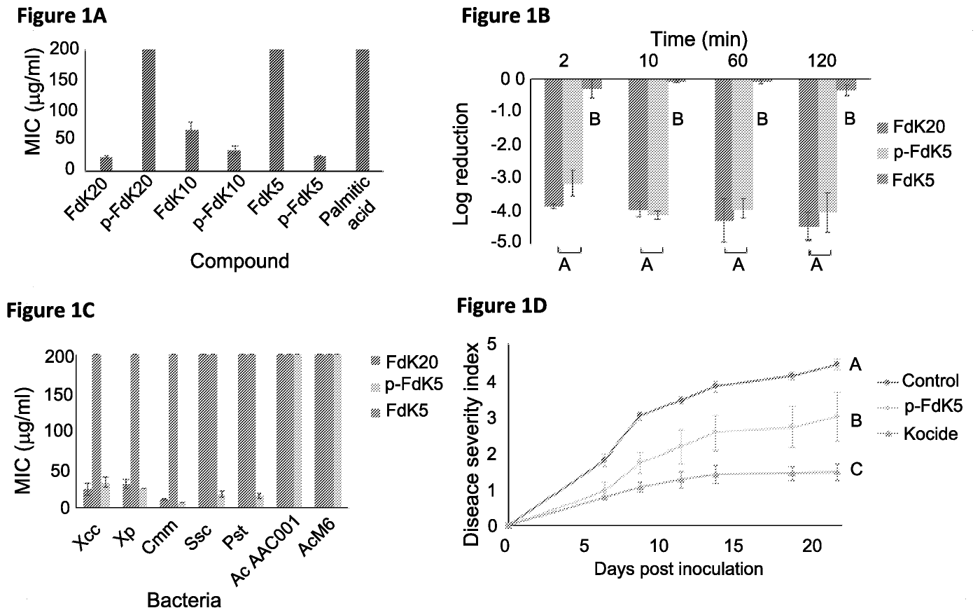

5-, 10- and 20-mer FdK RPMs were synthesized with or without palmitic acid acylation at the N-terminus amino group. Minimal inhibitory concentration (MIC) assays revealed that without the addition of palmitic acid, shortening the peptidic chain length compromised RPM antimicrobial activity as FdK10 was less active than FdK20, and FdK5 had no inhibitory activity (FIG. 1A).

In contrast, N-acylation of RPMs with palmitic acid (p-FdKn, where p refers to palmitic acid and n indicates the number of amino acids), showed that p-FdK10 had stronger inhibitory activity than FdK20. Moreover, the 5-mer lipo-RPM, p-FdK5, had strong inhibitory activity towards Xcc (FIG. 1A). Remarkably, p-FdK5 had similar antimicrobial activity as FdK20 (MIC values of 22 and 23 μg/ml, respectively).

The aforementioned findings were in agreement with results from bactericidal assays (FIG. 1B): p-FdK5 showed similar activity (p=0.924) as FdK20, as both reduced the initial Xcc population from ˜10⁸ to ˜10⁴ colony forming units (CFU)/ml after 10 min of incubation, while FdK5 showed no bactericidal effect over time. The inhibitory activities of RPMs and lipo-RPM were tested towards several important phytopathogenic bacteria (Table 2).

TABLE 2
Bacterial strains used in this study.

| Pathogen | Strain | Gram | Disease | Abbreviation | Source |
|---|---|---|---|---|---|
| Acidovorax citrulli | M6, | (−) | Bacterial fruit blotch | Ac M6, Ac | ^{[10]}; ^{[11]} |
| | AAC00-1 | | of cucurbits | AAC00-1 | |
| Xanthomonas | ATCC | (−) | Black rot disease of | Xcc | ^{[12]} |
| campestris pv. | 33913 | | crucifer plants | | |
| campestris | | | | | |
| Xanthomonas | 97-2 | (−) | Bacterial spot disease | Xp | ^{[13]} |
| perforans | | | of tomato and pepper | | |
| Pseudomonas | DC3000 | (−) | Bacterial spot disease | Pst | ^{[14]} |
| syringae pv. | | | of tomato | | |
| tomato | | | | | |
| Clavibacter | NCPPB | (+) | Bacterial canker and | Cmm | ^{[15]} |
| michiganensis | 382 | | wilt of tomato | | |
| subsp. | | | | | |
| michiganensis | | | | | |
| Streptomyces | Av | (+) | Potato common scab | Ssc | Burdman |
| scabies | | | | | lab |
| | | | | | collection |

p-FdK5 showed strong inhibitory activity against all tested strains except for the two tested strains of Acidovorax citrulli (Ac AAC00-1 and Ac M6; FIG. 1C). These results also demonstrate that p-FdK5 has broad antibacterial activity towards both Gram-positive and Gram-negative bacteria. Remarkably, while FdK20 did not show bacteriostatic activity towards Streptomyces scabies (Ssc) and Pseudomonas syringae pv. tomato (Pst), p-FdK5 showed strong inhibitory activity on these strains, which might suggest a different mode of action. p-FdK5 also showed great antimicrobial activity towards clinically isolated bacteria such as MRSA (data not shown).

To assess the potential of p-FdK5 as crop protection agent, we carried out in planta experiments using the Xanthomonas perforans-tomato pathosystem. Xanthomonas perforans is the causal agent of bacterial spot disease, one of the major diseases of tomato in many parts of the world. No visible phytotoxic effects were observed in leaves treated with p-FdK5 (not shown). Pretreatment of tomato leaves with p-FdK5 significantly (p<0.0001) reduced bacterial spot symptoms relative to non-treated controls (FIG. 1D). The reduction of disease severity by p-FdK5 was significantly less effective than pretreatment with the copper hydroxide-based commercial bactericide Kocide® 2000 (p<0.0001). However, it is important to note that the applied concentration of p-FdK5 was 12.5-fold lower than that of Kocide (200 and 2500 μg/ml, respectively). The hypothesis was that the efficiency of the lipo-RPM might be further optimized by application at higher concentrations and/or development of an appropriate formulation.

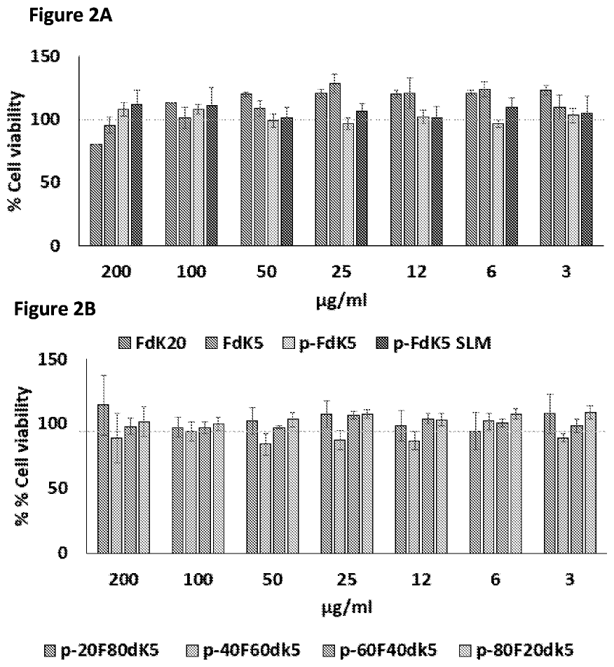

Some AMPs display characteristic selectivity, favoring attack on prokaryotic membranes relative to eukaryotic ones. However, native lipopeptides tend to be less selective, being quite toxic to mammalian cells. Cytotoxicity assays with HEK293T-2 mammalian cells revealed that FdK5 and p-FdK5 did not exert any toxic effects at 200 μg/ml. In contrast, exposure to 200 μg/ml of FdK20 led to a reduction of about ˜20% in viability of these cells (FIG. 2A). Therefore, we conclude that reduction of the peptidic chain in combination with addition of palmitic acid improves the selectivity of the mixture towards bacterial cells relative to tested mammalian cells.

p-FdK5 is a short, 5-mer lipo-RPM that contains 32 possible short lipopeptides in the mixture (2⁵; see all possible sequences in Table 3).

TABLE 3
Lipopeptides, RPMs and lipo-RPMs used in this study.

| | | | | % ACN at | |
|---|---|---|---|---|---|
| | | Predicted | Observed | retention | Average |
| #¹ | Peptide² | MW³ | MW³ | time | MIC |
| 1 | p-kkkkk | 896.31 | 896.46 | 34 | 6 |
| 2 | p-kkkkF | 915.32 | 915.51 | 37 | 6 |
| 3 | p-kkkFk | 915.32 | 915.54 | 37 | 4 |
| 4 | p-kkFkk | 915.32 | 915.56 | 38 | 6 |
| 5 | p-kFkkk | 915.32 | 915.58 | 37 | 6 |
| 6 | p-Fkkkk | 915.32 | 915.6 | 38 | 12 |
| 7 | p-kkkFF | 934.31 | 934.56 | 43 | 14 |
| 8 | p-kkFkF | 934.31 | 934.51 | 42 | 6 |
| 9 | p-kFkkF | 934.31 | 934.54 | 43 | 5 |
| 10 | p-FkkkF | 934.31 | 934.5 | 44 | 6 |
| 11 | p-kkFFk | 934.31 | 934.45 | 42 | 6 |
| 12 | p-kFkFk | 934.31 | 934.45 | 42 | 12 |
| 13 | p-FkkFk | 934.31 | 934.51 | 42 | 6 |
| 14 | p-kFFkk | 934.31 | 934.55 | 42 | 8 |
| 15 | p-FkFkk | 934.31 | 934.6 | 43 | 10 |
| 16 | p-FFkkk | 934.31 | 934.68 | 44 | 50 |
| 17 | p-kkFFF | 953.32 | 953.6 | 51 | 50 |
| 18 | p-kFkFF | 953.32 | 953.61 | 51 | 50 |
| 19 | p-FkkFF | 953.32 | 953.61 | 51 | 38 |
| 20 | p-kFFkF | 953.32 | 953.59 | 60 | 100 |
| 21 | p-FkFkF | 953.32 | 953.57 | 51 | 38 |
| 22 | p-FFkkF | 953.32 | 953.53 | 52 | ND |
| 23 | p-kFFFk | 953.32 | 953.45 | 62 | 100 |
| 24 | p-FkFFk | 953.32 | 953.51 | 52 | ND |
| 25 | p-FFkFk | 953.32 | 953.54 | 51 | ND |
| 26 | p-FFFkk | 953.32 | 953.58 | 60 | ND |
| 27 | p-FFFFk | 972.32 | 996.52 | 70 | ND |
| 28 | p-FFFkF | 972.32 | 996.53 | 69 | ND |
| 29 | p-FFkFF | 972.32 | 996.56 | 67 | ND |
| 30 | p-FkFFF | 972.32 | 996.55 | 69 | ND |
| 31 | p-kFFFF | 972.32 | 996.53 | 68 | ND |
| 32 | p-FFFFF | 991.32 | 1015.5 | — | ND |
| — | FdK5 | 657.88-752.89 | 696-734 | — | ND |
| — | p-FdK5 | 896.31-991.31 | 916-954 | — | 18 |
| — | FdK20 | 2580.49-2960.53 | 2829 | — | 25 |
| — | p-20F80dk5 | 896.31-991.31 | 915-934 | — | 6 |
| — | p-40F60dk5 | 896.31-991.31 | 915-953 | — | 9 |
| — | p-60F60dk5 | 896.31-991.31 | 915-953 | — | 12 |
| — | p-80F20dk5 | 896.31-991.31 | 934-953 | — | 125 |

¹#, serial number.
²Sequence from N to C-terminus; p, palmitic acid.
³Predicted and observed MW in Da. All lipopeptides (except #32 which was not successfully purified under lab conditions), and lipo-RPMs were purified using HPLC and all lipopeptides/RPMs/lipo-RPMs were analyzed using MALDI-TUF. For RPMs and lipo-RPMs, the range of predicted MW and the observed main peaks MW are presented.
⁴ND, non-detected at tested concentrations (up to 200 μg/ml).

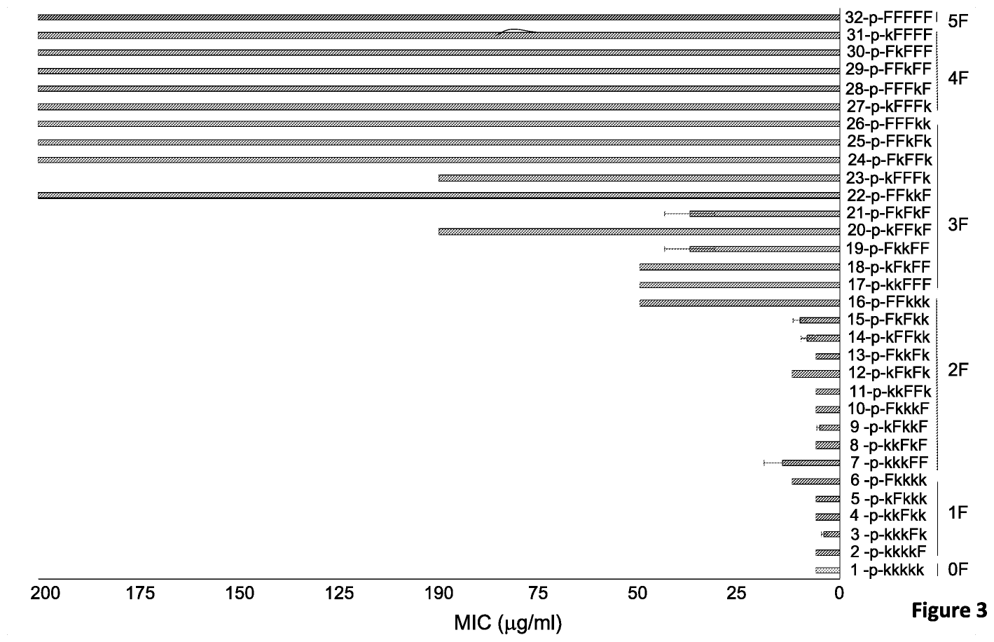

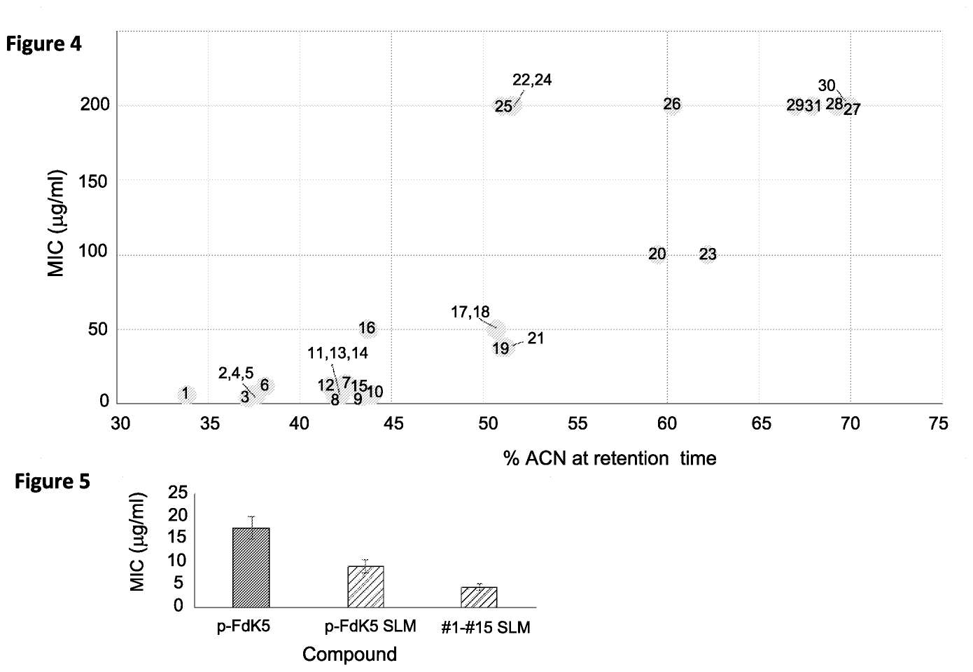

This fact provides a unique opportunity to perform mechanistic studies on the composition of individual sequence-specific lipopeptides, and to compare the antimicrobial activity of the mixture in comparison to individual peptides. 32 lipopeptides that compose the p-FdK5 mix were synthesized and tested individually in MIC assays (FIG. 3). Twenty two out of 32 (69%) lipopeptides were active against Xcc. The lowest MIC values (4-14 μg/ml) were attributed to lipopeptides containing 0 to 2 Phe (lipopeptide #1-15). Interestingly, the group of lipopeptides containing 3 Phe (#17-26) showed diverse MIC values: lipopeptides #22, 24 and 25 showed no inhibitory activity while others in this group showed MIC values ranging from 38 to 100 μg/ml. No inhibitory activity was observed for lipopeptides containing 4 or 5 Phe (#27-32). These results demonstrate that the ratio between hydrophobic and cationic amino acids is highly important in determining the antimicrobial activity of the lipopeptides. Another indication for this notion can be seen in the strong correlation between the MIC values and the levels of hydrophobicity of each lipopeptide as determined by the percentage of acetonitrile at the retention time in the HPLC purification (p<0.001, spearman Rho correlation coefficient=0.8704, FIG. 4). A previous study showed that the use of different molar ratios of Phe vs. Lys in synthesis of FK20 RPM greatly influenced the antimicrobial activity of the RPM. For example, a FK20 mixture that contained F:K at 1:9 ratio had significantly reduced antimicrobial activity relative to FK20 at a F:K ratio of 1:1. A higher hydrophobic ratio reduces activity.

In the case of p-FdK5, the reduced (or lack of) antimicrobial activity of lipopeptides that contain 3 or more Phe is likely due to the fact that palmitic acid strongly contributes to the hydrophobicity of the lipopeptides and these lipopeptides might aggregate.

It is further observed that the position of the hydrophobic amino acid within the lipo-RPM also plays a critical role in the antimicrobial activity. Lipopeptides containing Phe linked to and/or in a proximate position to the palmitic acid had the highest MIC values in their group. For example, lipopeptides #6 (p-Fkkkk) and #16 (p-FFkkk) showed the highest MIC values (12 and 50 μg/ml, respectively) within the group of lipopeptides containing 1 and 2 Phe, respectively. Similarly, lipopeptide #26 (p-FFFkk) was not active, while the other lipopeptides containing 3 Phe showed antimicrobial activity (as an example, the MIC value of lipopeptide #19, p-FkkFF, was 38 μg/ml). To conclude, the antimicrobial activity of the lipopeptides was influenced by both the ratio of hydrophobic vs. cationic amino acids and the position of the hydrophobic amino acids relative to the palmitic acid.

Interestingly, a mixture of equal amounts of the 32 purified lipopeptides, termed p-FdK5 specific lipopeptide mixture (SLM), showed a moderate difference in the MIC value as compared to the randomly synthesized p-FdK5 mixture (9 and 18 μg/ml, respectively, FIG. 5). Furthermore, #1-15 SLM, a mixture of equal amounts of all highly active lipopeptides with MIC values <15 μg/ml, showed higher inhibitory activity (MIC of 4 μg/ml) than the p-FdK5 lipo-RPM and p-FdK5 SLM (FIG. 5). This result can be probably explained by the fact that the #1-15 SLM lacks the less active lipopeptides present in the p-FdK5 RPM and in the p-FdK5 SLM.

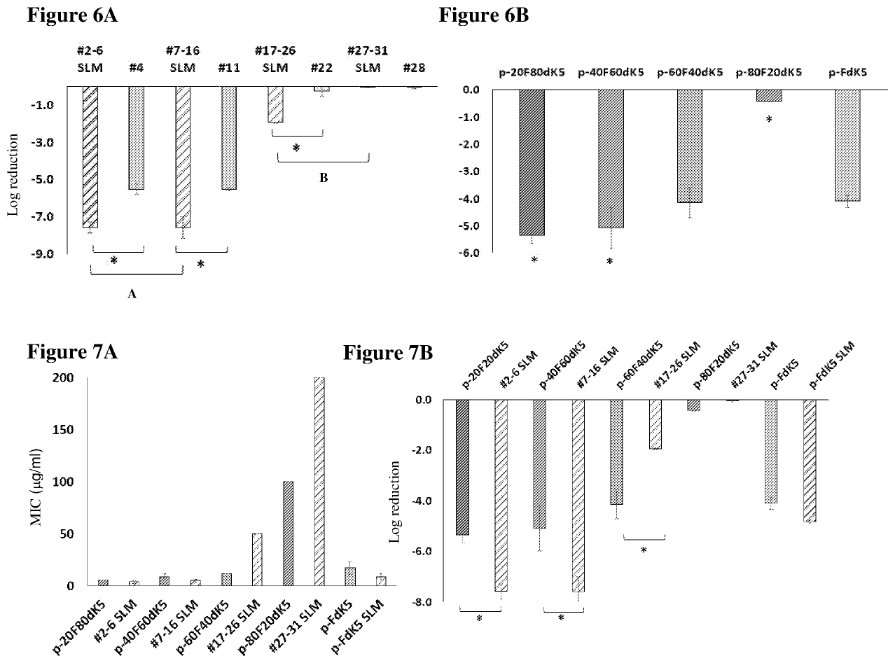

Further, different SLMs of lipopeptides were created with the same number of Phe (F) and D-Lys (dK) amino acids by mixing equal amounts of specific lipopeptides within each F:dK ratio group. For example, #2-6 SLM contained lipopeptides number 2 to 6, containing 1 Phe and 4 D-Lys residues. Bactericidal assay revealed that the less hydrophobic SLMs, #2-6 (1 F) and #7-16 (2 F), showed stronger bactericidal activity than the more hydrophobic ones, #17-26 (3 F) and #27-31 (4 F) (p<0.05, FIG. 6A), with log reductions of viable cells in the orders of ˜8, ˜7, ˜2 and ˜0, respectively. In addition, we compared each SLM with individual sequence-specific lipopeptide from each group to assess whether the mixtures have advantage over individual lipopeptides in terms of antimicrobial activity. Interestingly, in all cases the SLMs had significantly (p<0.05) higher bactericidal activities than the individual lipopeptides (FIG. 6A), thus suggesting the occurrence of synergistic interactions among lipopeptides within the mixture.

To assess whether the antimicrobial activity of the lipo-RPMs can be further improved, we synthesized lipo-5-mer RPMs using different ratios of F:dK in the synthesis solution (in contrast to the 1:1 ratio used for synthesis of the original p-FdK5). Bactericidal assays revealed that the lipo-RPMs synthesized with 20 and 40% Phe relative to D-Lys (p-20F80dK5 and p-40F60dK5, respectively) showed higher antimicrobial activity than original p-FdK5 (log reductions of ˜5 and ˜4, respectively; FIG. 6B). These lipo-RPMs were also more active than more hydrophobic mixtures that were synthesized with 60 and 80% Phe relative to D-Lys (p-60F40dK5 and p-80F20dK5, respectively). Growth inhibition assays showed a similar trend as p-20F80dK5 and p-40F60dK5 showed increased bacteriostatic activity than p-FdK5 (MIC values of 6 and 18 μg/ml, respectively; FIG. 7A). MTT assays revealed that, as similar as p-FdK5, these new lipo-RPMs had no cytotoxic effects (FIG. 2B).

Remarkably, growth inhibition assays also revealed that the #17-26 SLM (mixture of all lipopeptides containing 3 Phe) was less active than the p-60F40dK5 lipo-RPM (50 and 12 μg/ml, respectively). Similarly, while p-80F20dK5 had a MIC value of 100 μg/ml, the SLM containing equal amounts of all lipopeptides with 4 Phe (#27-31 SLM) was not active at all (FIG. 7A). Results from bactericidal assays were in agreement with results from growth inhibition assays: the relatively low hydrophobic SLMs #2-6 and #7-16 (composed by all lipopeptides containing one and two Phe, respectively) were significantly (p<0.001) more active than the randomly synthesized lipopeptide mixtures p-20F80dK5 and p-40F60dK5, respectively (FIG. 5B). In contrast, the relatively high hydrophobic SLM #17-26 SLM (3 Phe) was significantly less active (p<0.05) than the corresponding p-60F40dK5 mixture (FIG. 7B). These results are not surprising. For instance, when lipo-RPMs are synthesized in the presence of high F:dK ratio (60% and 80% phenylalanine relative to lysine), less hydrophobic lipopeptides can be generated thus increasing the antimicrobial activity of the mix in comparison with the corresponding SLMs that contain only highly hydrophobic lipopeptides.

The results strongly support the occurrence of synergistic interactions between individual lipopeptides in the mixtures (FIG. 6A). To further assess this assumption, a killing assay has been carried out, followed by calculation of the combination index (CI), and a checkerboard microdilution assay, followed by calculation of fractional inhibitory concentration index (FICi). These are the most common assays for assessment of the nature of interactions between different antimicrobial compounds. While the methodology of conducting these assays is well established, a variety of methods exists to interpret the results, which in turn can lead to different conclusions. Therefore, two different ways have been used to calculate these indexes (Table 4).

TABLE 4
Interactions between sequence-specific lipopeptides.

| | | | Checkerboard assay | |
|---|---|---|---|---|
| | Checkerboard assay | | Highest | Highest |
| | Average | Average | Single | Single |
| #lipopeptide | FICi | FICi | Agent | Agent |
| 2 + 5 | Indifference | Indifference | Synergy | Synergy |
| | (1.11) | (1.11) | (0.53) | (0.53) |
| 8 + 12 | Additivity | Additivity | Synergy | Synergy |
| | (0.92) | (0.92) | (0.83) | (0.83) |
| 18 + 23 | Indifference | Indifference | Synergy | Synergy |
| | (1.36) | (1.36) | (0.76) | (0.76) |
| 2 + 8 | Additivity | Additivity | Synergy | Synergy |
| | (0.82) | (0.82) | (0.45) | (0.45) |
| 2 + 18 | Indifference | Indifference | Synergy | Synergy |
| | (1.12) | (1.12) | (0.46) | (0.46) |
| 8 + 18 | Additivity | Additivity | Synergy | Synergy |
| | (0.94) | (0.94) | (0.77) | (0.77) |

The lowest FICi and the highest single agent index methods (for analyzing checkerboard and killing assays, respectively) support that there is a partially synergistic to a synergistic effect in most cases between sequence-specific lipopeptides in the mixture, independently of sequence and hydrophobicity. For instance, a synergistic effect was detected when combining lipopeptides #2 and #5 that belong to the same hydrophobicity group (lipopeptides composed by one Phe and four D-Lys residues). Similar results were obtained following combination of lipopeptides #2 and #8 (two Phe and three D-Lys residues). Overall, our findings demonstrate that lipo-RPMs have an advantage in terms of antimicrobial activity over a single active lipopeptide.

An additional advantage of RPMs and lipo-RPMs over single sequence-specific peptides/lipopeptides is that the formers are easier and cheaper to produce than the latter. Importantly, despite the potential of natural AMPs as antimicrobial compounds, it has been shown that bacteria are able to develop resistance to them by different mechanisms, such as decreased affinity through cell surface alterations, membrane efflux pumps and peptidases. For instance, both Escherichia coli and Pseudomonas fluorescens developed resistance towards pexiganan in an experimental evolution assay. However, a combination of pexiganan and melittin showed a slower resistance evolution as compared to application of individual AMPs and antibiotics in Staphylococcus aureus. Inventor's hypothesis is that the development of resistance to a cocktail of compounds is more difficult than to individual antimicrobial agents, and this is a critical advantage of RPMs and lipo-RPMs.

In summary, the potential of the lipo-RPM model p-FdK5 as a novel antimicrobial agent has been explored herein. p-FdK5 showed broad antimicrobial activity against Gram-positive and Gram-negative bacteria and had demonstrated selectivity against bacterial cells without causing cytotoxicity towards tested mammalian cells or plants. Moreover, p-FdK5 was able to reduce disease severity in the tomato-X. perforans pathosystem. Thorough analyses involving the 32 lipopeptides that compose p-FdK5 revealed that the antimicrobial effect is influenced not only by the ratio between hydrophobic and charged amino acids, but also by the dispersal of the hydrophobic residues along the peptide chain. Based on this notion new lipo-RPMs with improved antimicrobial activity were generated, demonstrating that the antimicrobial activity of the lipopeptide mixtures can be significantly improved by using relatively low ratios of hydrophobic versus cationic amino acids in the synthesized mixture. Lastly, the power of the mixture over single sequence-specific lipopeptides in terms of antibacterial activity has been demonstrated.

### Lipo-RPM's and Bacterial Growth

Antimicrobial random peptide mixtures (RPMs) have been recently proposed as powerful antimicrobial compounds. The inventors of the present disclosure have developed a new type of antimicrobial RPMs, lipo-RPMs. N-acylation of RPMs with palmitic acid enabled generation of short, 5-mer lipo-RPMs, with high and selective antimicrobial activity. Furthermore, all the 32 possible sequences embedded in the 5-mer lipo-RPM have been studies herein (25 optional sequences) and it is shown that the lipopeptide activity depends on the percentage of hydrophobicity and on the location of the hydrophobic amino acid relative to the palmitic acid. Improved 5-mer lipo-RPMs were generated by using different ratios of hydrophobic relative to cationic amino acids in the synthesis of the 5-mer lipo-RPMs. Also, synergism assays revealed positive interactions between different sequence-specific lipopeptides in terms of antimicrobial activity. The experimental data is presented herein-below:

### Experimental Procedures

Bacterial strains and growth conditions: The bacterial strains used in this study are detailed in Table 2. Unless stated otherwise, all strains were grown on nutrient agar (NA, Difco) or nutrient broth (NB, Difco), except Pst that was grown on Kings B agar plates and NB. All strains were grown at 28° C. for 48 h. All bacterial cells used in this study were stored in 25% glycerol at −80° C.

Synthesis of, random peptide mixtures (RPMs), lipo-RPMs and sequence-specific lipopeptides: All peptides were synthesized in SPE polypropylene single-Fritted tubes (Altech), on Rink amid resin (0.6 mmol/gr substitution, 0.1 mmol scale) using microwave irradiation (MARS: CEM, USA). Random peptide mixtures (RPMs) were synthesized according to Hayouka et al. (2013) using a modification of the solid phase peptide synthesis. Briefly, each coupling step was conducted with binary combinations of protected amino acids, with a freshly prepared stock solution containing the protected amino acids in the desired molar ratio between L-phenylalanine and D-lysine (2 equivalents from each amino acid, 0.2 mmol). Sequence specific peptides were synthesized according to the Fmoc solid-phase peptide synthesis method. The coupling reactions were conducted with 4 equivalents (0.4 mmol) of the needed pure protected amino acid. For generation of lipo-RPMs and sequence-specific lipopeptides, acylation was made by bounding palmitic acid to the N-terminus of the desired peptide/RPM using the same Fmoc chemistry of sequence-specific peptides, with the difference that overnight shaking at room temperature was used instead of microwave irradiation. Upon synthesis completion, RPMs/lipopeptides were cleaved from the resin [(95% Trifluoroacetic acid (TFA), 2.5% water, 2.5% Triisopropylsilane (TIPS)], re-suspended in double distilled water (DDW), frozen and lyophilized Evaluation of molecular weight was done using MALDI-TOF and amino acid content was quantified by amino acid analysis.

Purification of lipopeptides: Sequence-specific lipopeptides were dissolved in 20% acetonitrile in DDW or in DMSO and filtered before injection to reversed-phase high-performance liquid chromatography (RP-HPLC, Shimadzu) on a C18 column (Phenomenex 250×10.0 mm, 5 μm). Acetonitrile (Bio Lab) and 0.1% Trifluoroacetic acid (Bio Lab) in DDW were used as solvents. The molecular weight of the purified lipopeptides was validated by MALDI-TOF, then the lipopeptides were re-suspended in DDW, frozen and lyophilized.

Minimal inhibitory concentration (MIC) assay: MIC values were determined by broth microdilution in sterile 96-flat bottom well plates (Corning 3650) as described by Hayouka et al. (2013). Briefly, Xcc cells were grown for 24 h in NB at 28° C., with shaking (180 rpm) and re-suspended in fresh NB to reach an OD_{600 nm} of 0.1 (˜10⁸ CFU/ml) that was measured using a Genesys 10 uv spectrophotometer (ThermoSpectronic). One hundred microliter-aliquots of Xcc cells were added to 100 μl of NB medium containing peptides/lipopeptides at various concentrations. Bacterial growth was determined after incubation of 24 h at 28° C. by measurement of the optical density (OD_{595 nm}), using a Tecan infinite Pro Plate reader. MIC values were determined as the lowest concentrations that showed significant growth inhibition. The highest concentration tested was 200 μg/ml. Each experiment was carried out at least three times, with three replicates per strain/concentration combination in each experiment.

Bactericidal activity assays: Xcc bacterial cells were grown for 24 h in NB at 28° C., with shaking (180 rpm). Then the cells were washed with sterile phosphate buffered saline (PBS, 8 g/l NaCl, 0.2 g/l KCl, 0.6 g/l Na₂HPO₄, 0.2 g/l KH₂PO₄, pH 7.4) by centrifugation (3×2 min, 8000 rpm) and re-suspended in fresh PBS to OD_{600 nm} of 0.1 (˜10⁸ CFU/ml). Bacterial cells were then incubated (28° C., 180 rpm) with 25 μg/ml of the desired peptide/lipopeptide for different times, serially diluted in PBS and plated on NA. The plates were incubated at 28° C. for 48 h and bacterial colonies were counted to quantify the CFU/ml. The lower limit of sensitivity of colony counts was 10 CFU/ml. Each experiment was carried out three times, with two replicates per treatment in each experiment. Data were statistically analyzed by non parametric comparison for all pairs using Dunn method for joint ranking (alpha>0.05), using JMP Pro 12 (SAS Institute).

In planta assays: The ability of p-FdK5 to reduce disease severity using the tomato-Xp pathosystem (bacterial spot disease) was assessed by the inventors. The experiments were carried out as described. Briefly, tomato (Solanum lycopersicum) cv. FA-144 plants were used for inoculation after they developed four fully expanded leaves (˜4 weeks after transplant). The five most external leaflets of the 3 youngest fully expanded leaves were pre-treated on both sides with solutions containing p-FdK5 (200 μg/ml), Kocide® 2000 (2500 μg/ml; DuPont) or sterilized DDW (control). Twenty-four hours later each leaflet was sprayed on both sides with a bacterial suspension containing ˜5×10⁸ CFU/ml of X. perforans 97-2 (OD₆₀₀ of 0.2). Inoculated plants were covered with a plastic bag for 24 h to promote disease. Disease severity in each leaflet was determined every two to three days after appearance of disease symptoms [˜5 to 6 days post inoculation (dpi)], using a 0-6 scale. Assessment of phytotoxic effects on treated leaves was done by following appearance of symptoms in plants that were not inoculated with bacteria, but rather sprayed with sterile DDW. Three experiments were carried out. In each experiment, each treatment was applied on 5 plants, 3 leaves per plant, 5 leaflets per leaf. Data were statistically analyzed by ANCOVA (dpi as covariant) using JMP Pro 12 (SAS Institute).

Cytotoxicity towards mammalian cells: HEK293T (ATCC number CRL-11268) cells were grown at 37° C. in a humidified atmosphere with 5% CO₂ in Dulbecco's modified Eagle's medium (DMEM, Sigma) with 2 mM L-glutamine, 1% (v/v) Penstrep and 20% (v/v) fetal bovine serum (Biological Industries). The cytotoxic potential of RPMs/lipo-RPMs was determined using the MTT assay, as described. Briefly, one hundred microliter aliquots of 5×10⁵ viable cells/ml were transferred to wells in a 96-well microplate (Nunc, Thermo Scientific) and incubated at 37° C. After 24 h the cells were washed, re-suspended in DMEM without phenol and incubated with tested RPMs/lipo-RPMs at two-fold serial dilutions, Tween or Triton-X (as positive controls), and PBS or DMEM (as negative controls). After incubation of 24 h, the cells were washed by removing the media by suction and replacing it with a fresh media containing 50 μl of 3-(4,5-dimethylthiazol-2-yl)-2,5-diphenyltetrazolium bromide (MTT, Sigma) solution in culture media (0.5 mg/ml) and left for incubation for 90 min. The media were removed by suction, then 100 μl of dimethyl sulphoxide (DMSO) were added to each well. Plates were incubated in dark conditions for 10 min at 37° C. and optical density (OD_{595 nm}) was measured using a Tecan Infinite Pro plate reader. The percentage of viable cells was calculated by comparison of treated cells to control cells. Experiments were carried out three times, with four replicates per RPM/lipo-RPMs concentration in each experiment.

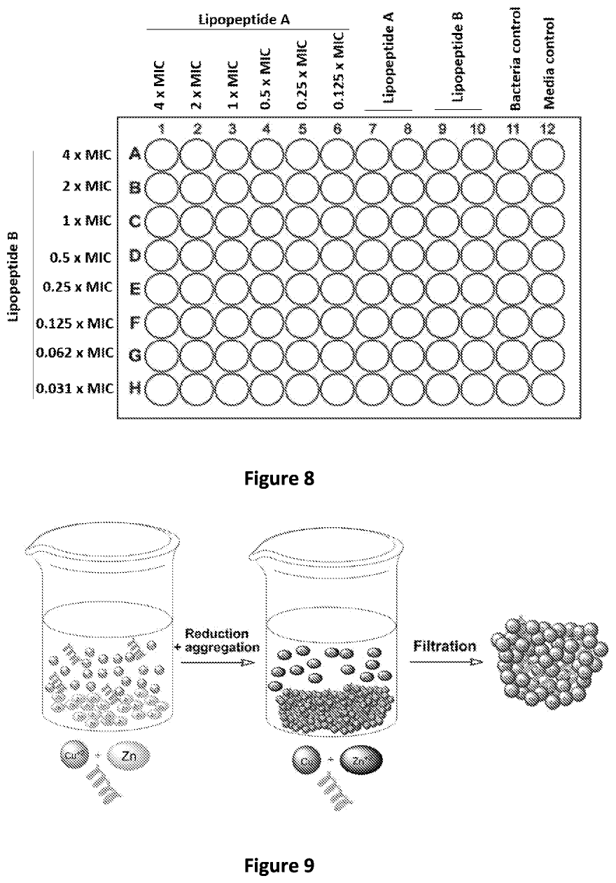

Synergy assessment using the checkerboard assay: The checkboard assay was used to assess possible synergistic interactions between sequence-specific lipopeptides. Xcc cells were grown for 24 h in NB at 28° C. with shaking (180 rpm) and re-suspended in fresh NB to reach an OD_{600 nm} of 0.1 (˜10⁸ CFU/ml). MIC assays were conducted in sterile 96-well flat bottom well plates (Corning 3650) containing two-fold dilutions of the two tested lipopeptides combined in different concentrations starting from 4×MIC values (columns 1-6). For each tested lipopeptide, two columns containing two-fold serial dilutions of the tested lipopeptide alone, starting at concentrations of 4×MIC were used to evaluate the MIC (columns 7-10), while the remaining columns were used as positive control (wells containing bacteria with NB without tested lipopeptides) and negative control (wells containing NB without bacteria, columns 11 and 12, respectively, FIG. 8). The plates were incubated for 24 h at 28° C. and bacterial growth was determined be measurement of optical density (OD_{595 nm}) using a Tecan infinite Pro plate reader. Three independent experiments were carried out for each lipopeptide combination, with two replica plates in each experiment. The interpretation of the checkerboard synergy test results was done using the Fractional Inhibitory Concertation index (FICi) according to the following equation:

$${FIC i}_{A B} = \frac{{MIC}_{A}^{Comb}}{{MIC}_{A}^{Alone}} + \frac{{MIC}_{B}^{Comb}}{{MIC}_{B}^{Alone}} , \qquad Eq . 1$$

where MIC_{A}^{Comb} is the MIC of lipopeptide A when combined with lipopeptide B, and MIC_{A}^{Alone} is the MIC of lipopeptide A alone, without the presence of compound B.

Two methods were used to determine the FICi: 1) the Mean FICi, which considers the concentrations in the first non-turbid (clear) well found in each row and column along the turbidity/non-turbidity interface and then averaged; 2) the Lowest FICi, which is the lowest FICi of all non-turbid wells along the turbidity/non-turbidity interface. The FICi obtained was interpreted as follows: <0.5 denoting synergy; 0.5-0.75 denoting partial synergy; 0.76-1 denoting an additive effect; 1-4 denoting indifference; and >4 denoting antagonism.

Synergy assessment using the killing assay: The killing assay was used as an additional approach to assess possible synergistic interactions between sequence-specific lipopeptides. It was carried out as similar as described above in ‘Bactericidal activity assays’, but in these experiments Xcc cells were incubated (28° C., 180 rpm) for 10 min with 1×MIC concentration of each lipopeptide or with a combination of two lipopeptide at 1×MIC concentration each. The Combination Index (CI) method was used to assess the nature of the interactions between the lipopeptides. CI is the standard index used to reflect the combination effect, which can be greater (CI<1), lesser (CI>1) or similar (CI=1) to the expected additive effect of the two compounds being tested. CI was calculated in two different ways: 1) Using the Highest Single Agent approach, in which the CI is determined as:

$$CI = \frac{max ( {E}_{A} , {E}_{B} )}{{E}_{AB}} , \qquad Eq . 2$$

where E_{A} is the effect observed by one of the lipopeptides, E_{B} is the effect observed by the second lipopeptide, and E_{AB} is the effect caused by combining the two lipopeptides; and 2) Using the response additivity approach in which the CI is calculated as follows:

$$CI = \frac{{E}_{A} + {E}_{B}}{{E}_{A B}}$$

Experiments were carried at least three times, with two replicates per treatment in each experiment.

Statistical analysis: Statistical analysis was done using JMP Pro 15 (SAS Institute). Data from bactericidal assays (FIG. 1B), bacteriostatic activity towards Xcc of RPMs and MSL with different phenylalanine:D-lysine ratios (FIG. 7B), comparison between different p-FdK5 SLMs with individual lipopeptides from each group (FIG. 6A) data were statistically analysed by non parametric comparison for all pairs using the Dunn method for joint ranking (alpha=0.05). The effect of p-FdK5 on bacterial spot disease of tomato (FIG. 1C) was statistically analysed by ANCOVA (dpi as covariant, with blocking effect, alpha=0.05).

Comparison among 5-mer lipo-RPMs that were synthesized using varying concentrations of F and k (FIG. 6B) data was analyzed by non parametric comparison to p-FdK5 treatment using Dunn method for joint ranking (alpha=0.05).

Correlation between the percent of acetonitrile at retention time of specific lipopeptides and the mic value against Xcc (FIG. 4) was statistically analysed by the Spearman Rho correlation coefficient, alpha=0.05).

### Compositions of the Invention

Materials and Methods

Chemicals and bacterium: Fmoc-L-leucine-OH, Fmoc-L-lysine(Boc)-OH, 2-(1H-benzotriazole-1-yl)-1,1,3,3-tetramethyluronium hexafluorophosphate (HBTU), and rink amide resin (0.53 mmol/gr) were purchased from Chem-Impex (USA). Sodium chloride, N,N-dimethylformamide (DMF), diethylether, trifluoroacetic acid (TFA), and N,N-diisopropylethylamine (DIEA) were purchased from Biolab, Israel. Yeast and tryptone were obtained from BD (Franklin Lakes, N.J.). Copper sulfate, zinc (granular) and all other chemicals were purchased from Sigma Aldrich. The bacterium methicillin resistant Staphylococcus aureus 1206 was obtained from Prof. B. Weisblum (UW-Madison, Wis., USA).

Random peptide mixtures synthesis: Random peptide mixtures were synthesized using microwave irradiation on rink amide resin (Substitution 0.53 mmol/gr, 25 μmol) in filter tubes (Silicol) as described previously. Briefly, coupling reactions were conducted with binary combinations of Fmoc-L-Leucine-OH and Fmoc-L-Lysine(Boc)-OH. A freshly prepared stock solution that contained the protected amino acids in 1:1 molar ratio was used for each reaction. Prior to each coupling step, an aliquot containing 4 equivalents (100 μmol) of the amino acid mixture was activated with 4 equivalents of HBTU, and 8 equivalents of DIEA in DMF and added to the resin. The reaction mixture was then heated to 70° C. in a MARS VI (CEM, USA) multimode microwave (2 minutes ramp to 70° C., 4 minutes hold at 70° C.) with stirring. For Fmoc deprotection we added 20% piperidine in DMF and heated the reaction solutions to 80° C. in the multimode microwave (2 minutes ramp to 80° C., 3 minutes hold at 80° C.) with stirring. After each coupling/deprotection cycle, the resin was washed 3 times with DMF. At the end of the synthesis, the peptide mixtures were cleaved from the resin by stirring in a solution containing 95% trifluoroacetic acid (TFA), 2.5% double-distilled water (DDW), and 2.5% triisopropylsilane for 3 hours. The peptides were then precipitated by the addition of cold ether and centrifuged. The ether was then removed, and the peptide pellet was dried under a stream of nitrogen, frozen in liquid nitrogen, and lyophilized. The synthesis was validated by MALDI TOF MS. The resulting peptide mixtures composed of Leucine-lysine termed LK 20-mer or LK 10-mer, depends on the chain length.

Composite preparation: CuSO₄ (0.200 g, 1.25×10⁻³ mol) was dissolved in 2.0 mL double distilled water (DDW). Zinc powder (0.081 g, 1.25×10⁻³ mol) was added and then after 30 s of stirring, 1.0 mL of a peptide solution (0.003 g, LK 20 or 10-mer) was added at a 50:1 ratio of copper to amino acid. The solution was mixed for 24 h at room temperature (˜23° C.). The resulting precipitate was filtered through sinter glass (pore size of 10-16 μm), washed with 30 mL of DDW and dried overnight under vacuum. Non-doped copper composite was prepared by the same procedure without adding any peptides.

Measurement of copper ion concentration: The copper ion concentration was measured as follows: 10 mg of the composites were dispersed in 50 mL Luria broth (LB) growth media at 37° C. and shaken at 180 rounds per minute (RPM) for 8 hours. After this time, a 5.0 mL sample was removed from the suspension, filtered through a 0.45 Inn filter, and digested (4.5 mL) with 3.0 ml of 65% HNO₃ and 2.0 mL of 30% H₂O₂. The samples were completely dissolved for analysis and measured by inductively coupled plasma mass spectrometry (ICP-MS).

Growth inhibition assays: LB inoculated with MRSA was incubated overnight at 37° C. with shaking at 180 RPM. A bacterial suspension with optical density (O.D.) of 0.1 at 600 nm was then prepared and diluted 1000-fold in LB (to approximately 10⁵ CFU/mL). The tested composites (10 mg, 200 ppm) were added to sterile 100 mL Erlenmeyer flasks, and 50 mL of MRSA bacterial suspension was added. The flasks were incubated at 37° C. with shaking at 180 RPM for 24 hours and bacterial growth was monitored by measuring the O.D._{600 nm} of 1.0 mL samples removed at different time points. Flasks without composites or with leucine-lysine random peptides mixtures (LK) (4.0 μg/mL) were used as negative and positive controls, respectively. The percentage of growth inhibition was calculated at each time point (Tᵢ) according to the equation:

$$\% Growth Inhibition ( {T}_{i} ) = 100 - \frac{O . D . treatment at {T}_{i}}{O . D . negative comtrol at {T}_{i}}$$

Mixing Leucine-Lysine random peptide mixtures and copper ions assay: MRSA cells were grown as described in order to prepare a bacterial suspension with O.D._{600 nm} of 0.1. Aliquots (1 mL) of this bacterial suspension were mixed with an equal volume of LB containing copper sulfate and various concentrations of LK 20 or 10 mer peptides to give final concentrations of 400 ppm copper sulfate with 25 or 12 μg/mL LK20-mer, or with 200 or 100 μg/mL LK10-mer. After mixing by vortex, a 1 mL sample was taken for O.D. measurement and the remaining 1 mL sample was incubated for 6 hours at 37° C., 100 RPM. After this time, the O.D. was re-measured and the percentage growth was calculated according to the equation:

$$\% Growth ( {T}_{i} ) = \frac{O . D . {T}_{6 h treatment} - O . D . {T}_{0 h treatment}}{O . D . {T}_{6 h control} - O . D . {T}_{0 h control}} \times 100$$

Assessment of peptide release: Two methods were used to examine the release of peptides from the composites. (I) Composites (10 mg) were suspended in 50 mL DDW and incubated for 6.5 hours with shaking at 37° C., 180 RPM. At the end of this incubation time, the suspension was filtered through sintered glass (size pores of 10-16 μm). The filtered composite was then dried under vacuum and the organic elements content was analyzed as described above. A lack of change in the weights of the elements indicated that the peptides were not released from the composite. (II) The filtered solution was freeze-dried, then suspended with 80 μl DDW and the protein content of a 50 μl aliquot was analyzed by the addition of 450 μl of 4-fold diluted Bradford reagent. After 5 minutes incubation in the dark, the peptide content was measured by reading the O.D. at 595 nm against a standard curve prepared with serial dilutions of free LK random peptides in DDW. The linear range was 6-100 μg/mL.

Instrumentation: Peptides synthesis was validated by MALDI TOF MS Microflex LRF (Bruker Daltonik GmbH, Germany) UV-Vis absorbance spectroscopy was carried out with Genesys 10 uV UV-vis spectrophotometer (Thermo Spectronic). Elemental analysis (nitrogen, carbon, hydrogen, and sulfur) of at least 4 different batches was carried out using a Thermo Elemental Analyser 1120. Thermogravimetric analysis (TGA) from 50° C. to 800° C. was conducted with a Mettler-Toledo TGA/SDTA 851e, at a heating rate of 10° C. per minute under N₂ atmosphere. Density measurements were carried out with a Micromeritics AccuPyc 1340 instrument using helium as the displacing gas. The copper ion concentration was measured by an axial inductively coupled plasma optical emission spectrometer (ICP-OES) model ‘ARCOS’ from Spectro GMBH (Germany). SEM (scanning electron microscope) and EDAX (energy-dispersive X-ray spectroscopy analysis) were carried out on a Sirion (FEI) high resolution (HR) SEM instrument. X-ray powder diffraction measurements were performed on a D8 Advance diffractometer (Bruker AXS, Karlsruhe, Germany) with secondary Graphite monochromator, 2° Sollers slits, and 0.2 mm receiving slit. The powder samples were placed on low background quartz sample holders. XRD (X-ray diffraction) patterns from 5° to 85° 20 were recorded at room temperature using CuKα radiation (λ=0.15418 nm) under the following measurement conditions: tube voltage of 40 kV, tube current of 40 mA, step scan mode with a step size of 0.02° 2θ and a counting time of 1 s per step. The Scherrer equation was used to obtain the crystallite size from the experimental XRD data. The instrumental broadening was determined using LaB6 powder (NIST SRM 660).

Results:

Leucine-lysine random peptide mixtures with two different chain lengths of 10 and 20 amino acids (termed LK 10-mer and LK 20-mer), were synthesized, using modified Fmoc-solid phase peptide synthesis methodology. Synthesis success was validated by mass spectrometry analysis. Copper was doped with the random peptide mixtures by a modified version of the heterogeneous doping methodology which is based on the reduction of the copper cation with metallic Zn (see Experimental details). A control of copper ([Cu]) was prepared by the same procedure, but in the absence of the random peptides.

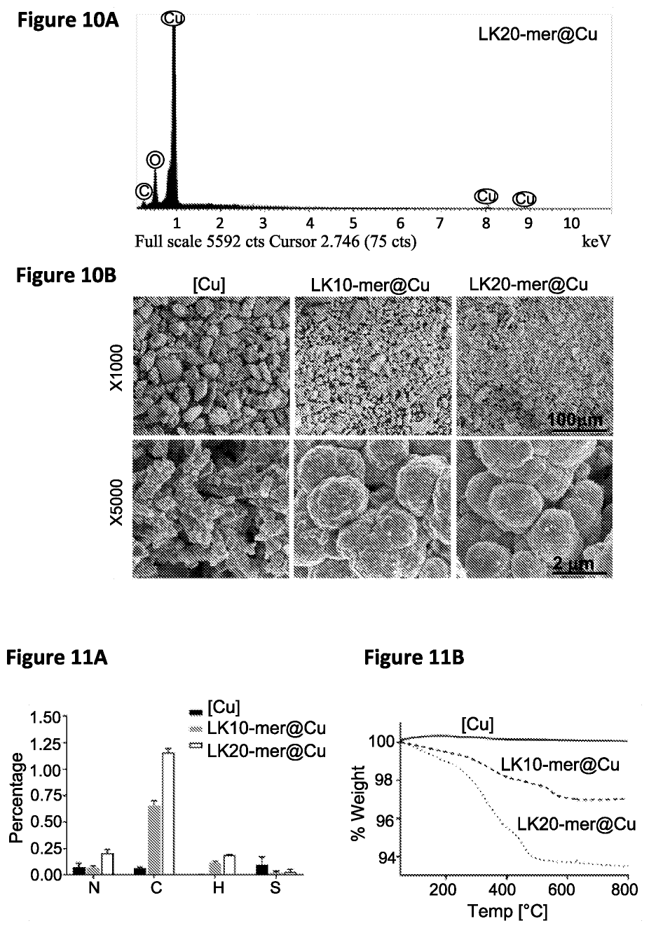

The new materials were characterized by several chemical and physical measurements. The densities of the LK20-mer@[Cu] and LK10-mer@[Cu] were lower (7.2 and 7.3 g/cm³, respectively) than of the non-doped copper composite (8.0 g/cm³) as the peptides interfere the growth of copper crystals along the reduction process. Energy dispersive X-ray analysis (FIG. 10A) showed that the composites contained only copper and organic material, with no detectable traces of zinc. The use of a lower copper to zinc ratio (1:1.2), reveals residual traces of zinc indicating that the 1:1 ratio of copper to zinc is optimal for successful reduction without any residual contamination of zinc. The structure and morphology of the composites were characterized by scanning electron microscopy (SEM) (FIG. 10B). Nanocrystals of copper tightly aggregated to form micron sized microparticles, which are aggregated to form large clusters. Interestingly, as observed by the X1000 magnification (FIG. 10B, upper row), the peptide entrapment appears to reduce the size of the particles with the longer chain random peptides resulting a smaller particle. This reduction in size might be attributed to inhibition of nanocrystal growth caused by the adsorptive interactions of the LK RPM with the copper.

Elemental analysis confirmed the entrapment of the random peptides in the copper matrix (FIG. 11A). The results provide qualitative evidence for the presence of organic elements in the LK@[Cu] composites as opposed to the undoped copper. Higher percentages of nitrogen and carbon (0.21% and 1.16%) were detected in the LK20-mer@[Cu] composites than in the LK10-mer@[Cu] composites (0.07% and 0.66%).

Thermal gravity analysis was performed to quantify the amount of the entrapped organic material (FIG. 11B), measured a 2.98% weight loss for the LK10-mer@[Cu] and 6.51% for the LK20-mer@[Cu]. X-ray Powder Diffraction (XRD) measurements revealed that the composites contains cuprite (Cu₂O) as well as metallic copper. The copper ([Cu]), LK10-mer@[Cu], and LK20-mer@[Cu] contained 25.8%, 33.1 and 44.1% cuprite, respectively. The results indicate that the random peptides entrapped in a matrix composed of not only a copper but copper and cuprite both, therefore the matrix termed as [Cu] along the application.

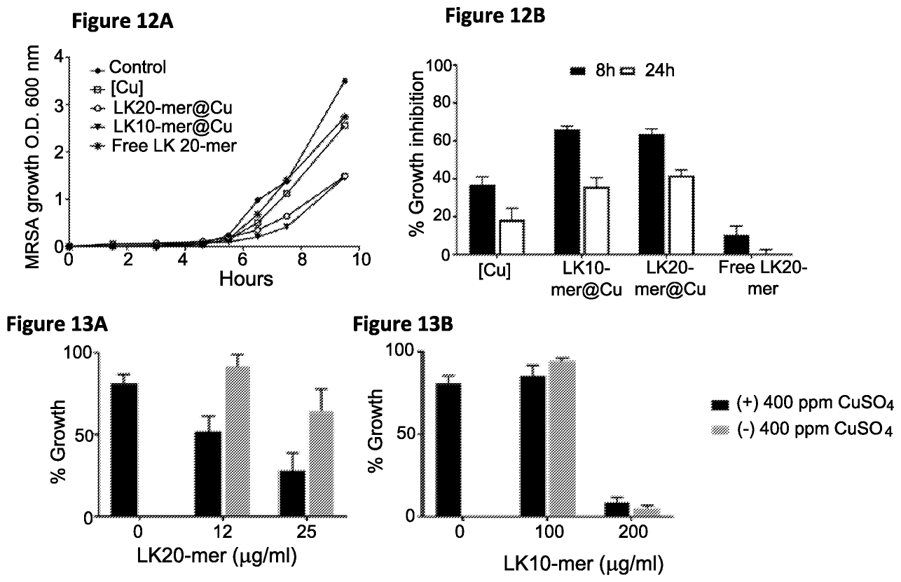

The next step was to evaluate the biological activities of the composites. FIG. 12 shows the effect of the composites on the growth of MRSA bacterial cells. While copper ([Cu]) had only a minor effect on MRSA growth, the entrapped peptide composites at the same concentration strongly inhibited the bacterial growth. This inhibition was maintained over the course of the culture, with a narrower gap after 24 hours. As presented in FIG. 12, the free LK 20-mer random peptide that was used as a control (at 4 μg/mL, in accordance with the estimated maximal amount of peptide in 200 ppm composite), did not inhibit bacterial growth.

Since the two components displayed a synergistic effect, it was important to evaluate the release of copper ions from the composites. Copper ions were released from the composites in a liquid medium according the oligodynamic effect. After 8 h incubation in growth medium no significant differences was detected by inductively coupled plasma mass spectrometry (ICP-MS) in the amount of copper ions that were released from the LK@[Cu] composites compared to the nondoped composite [Cu]. As for the peptides (by using Bradford assay)—there was no detectable release of peptides from the composites, even after extended incubation. To verify this finding, we also performed elemental analysis of composites before and after incubation in water. The results showed only a slight-zero weight decrease, supporting the observation that there was no significant release of random peptides during the incubation of the composites.

In order to evaluate the benefits of generating the LK@[Cu] composites compare to a simple physical mixing of the two active agents, the activity of copper sulfate and LK peptides after mixing both agents was tested. As presented in FIG. 13, copper ions have a weak effect on the growth of MRSA (˜15% lower than the control). The free LK20-mer peptide had a concentration-dependent inhibitory effect (Grey bars), which was enhanced in the presence of copper ions (Black bars). In contrast, addition of copper ions to LK10-mer did not enhance the antimicrobial activity. These findings support that physical mixing of LK and copper ions is not enough to achieve a synergistic antimicrobial effect as the composites were shown.

### Discussion

The LK@[Cu] composites were generated by the entrapment of random peptide mixtures in a copper-cuprite matrix represent a new material with unique properties and characteristics, rather than being simply the sum of the component parts separately. The composites were prepared according to the heterogeneous reduction method with zinc as the reducing agent where copper agglomerates to microcrystals. Random peptides that interact with the copper entrapped between the aggregates, following which the zinc is washed away as confirmed by the EDAX analysis. Evidence for the formation of a new composite material is given by the reduction in the density and particle size after entrapment of the peptides. This is in agreement with the trend of decreasing densities of other composites (Nafion@Cu, Thionin@Cu) and may be attributable to disturbances in the organization of a typical crystal structure. The absence of detectable release of peptides from the composites also suggests the formation of strong and specific interactions between the LK random peptides and the copper in addition to the physical entrapment. This is in accordance with reports that large molecules such as Nafion and the enzyme acid phosphatase were not released from composites although small molecules such as chlorohexidine or analgesics were entrapped in copper or silver were able to be released. According to the results of the TGA and elemental analysis, the LK 20-mer random peptides were entrapped more efficiently than LK 10-mer random peptides. Since copper competes with water for interaction with the peptides, we hypothesize that a longer peptide chain length, with a higher surface area, has a greater probability to interact with the copper and be entrapped.

In addition to the physical attributes, the composites also displayed unique properties with stronger antimicrobial activity against MRSA than would be expected from the total sum of the constituents. This activity is due to synergism of the two components and was not seen when each component (copper ions and LK 10 or 20 mer peptides) was tested alone; or when combined by physical mixing. These results emphasize that the entrapment formed a new material with its own antimicrobial activity that both the copper and cuprite have a role in addition to the LK RPM.

An interesting observation was that while previous work of the inventor indicated that higher concentrations of the LK 10-mers were needed for growth inhibition of bacteria than LK 20-mer random peptide mixtures (FIG. 13), the results presented here showed that both the LK10-mer@[Cu] and LK20-mer@[Cu] composites had a similar activity. The reason for this discrepancy is not entirely clear although it probably related to the structure of the composites. Since no release of peptides from the composite was detected, we assume that LK@[Cu] is active in the entrapped state and the external extruded parts of the 10-mer or 20-mer peptides chains left available for interactions, represent an “antimicrobial bioactive surfaces”. In a liquid solution, according to the oligo-dynamic effect, metal ions are slowly released, followed by a loosening of the tight structure, which may expose the internal random peptides to the bacteria. The suggested mode of action of the composites is that when the bacteria approach the surface of the composites, the cationic random peptides attract and disrupt the bacterial membrane permitting the entry of copper ions into the bacterial cells.

### Lauroyl Arginine Ethyl and Copper Composites

Synthesis of composites: To a solution of copper sulfate (CuSO₄) LAE and Zn were added. Zn was added at an equal molar ratio as Cu in the solution (1:1 ratio). Zn acts as a Cu reducing agent.

The reaction is shows herein:

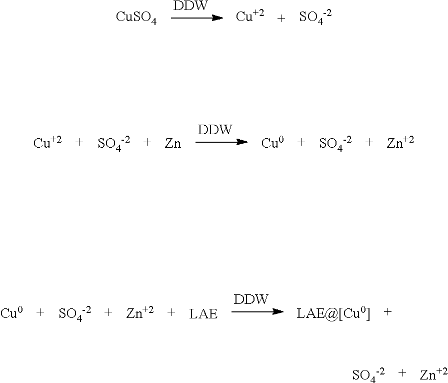

After incubation at 25° C. for 24 h, the resulting composites are filtrated with a 0.22-μm membrane and dried under vacuum overnight at room temperature.

Synthesis of different composites using different amounts of LAE is shown is Table 5 below:

TABLE 5
Reaction characteristics for synthesis of different types
of LAE@Cu composites.

| | | | | | LAE | |
|---|---|---|---|---|---|---|
| | | | | | concen- | % of LAE |
| | CuSO₄ | LAE | Zn | DDW | tration | entrap- |
| Composite ¹ | (mg) | (mg) | (mg) | (ml) | (mM) ² | ment ³ |
| LAE × 1.0@Cu | 200 | 5.2 | 81 | 3 | 4.12 | 5.0 |
| LAE × 0.5@Cu | 200 | 2.6 | 81 | 3 | 2.06 | 4.5 |
| LAE × 0.25@Cu | 200 | 1.3 | 81 | 3 | 1.03 | 1.0 |
| @Cu | 200 | 0 | 81 | 3 | 0 | 0 |

¹ three different composites were synthesized. For synthesis of LAE × 1.0@Cu we used the maximal amount of LAE in the solution. For synthesis of LAE × 0.5@Cu and LAE × 0.25@Cu composites, we used ½ and ¼ the amounts of LAE as used for synthesis of LAE × 0.5@ Cu composites. For synthesis of @Cu (control), no LAE was added.
² LAE concentration (mM) in the incubation solution.
³ Percentage of LAE entrapment [% (w/w) relative to Cu] in the composites as determined by different methods (see below).

Characterization of the different types of LAE@Cu composites: Chemical characterization of the synthesized composites was accomplished by different tests as described below.

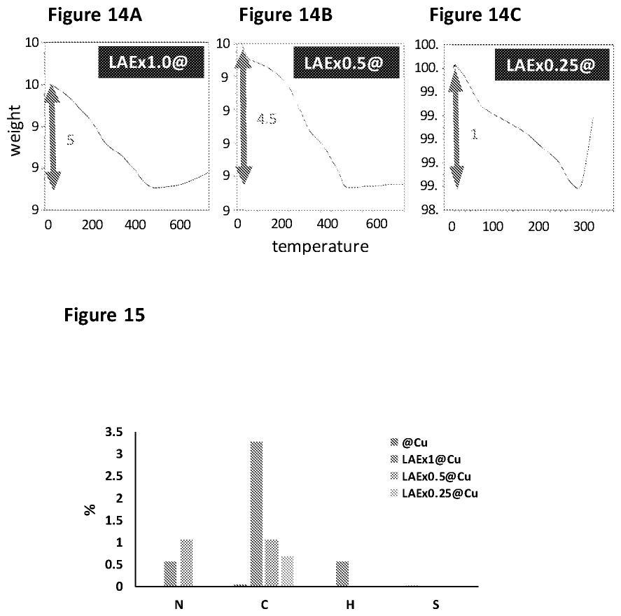

Thermal gravimetric analysis (TGA): This technique allows determining the maximal amount of the organic compound entrapped within the composite. In this assay, the composites are heated to gradually increasing temperatures up to 800° C. During this process the organic compounds are burnt. As consequence, there is a reduction of the dry weight of the composite relative to the initial composite weight. This reduction can be measured, thus allowing quantification of the initial concentration of the entrapped molecules. These experiments allowed us to conclude that composites LAE×1.0@Cu, LAE×0.5@Cu and LAE×0.25@Cu have entrapped LAE at concentrations [% (w/w)] of 5%, 4.5% and 1%, respectively (Table 5, FIG. 14).

Elemental analysis: This analysis allows quantification of the elements carbon (C), nitrogen (N), hydrogen (F) and sulfur (S). Since the first three elements are present in LAE, this test serves as an additional analysis to quantify the amount of LAE entrapped within the Cu matrix. Results from this analysis are shown in FIG. 15 and indicate that LAE×1.0@Cu, LAE×0.5@Cu and LAE×0.25@Cu and @Cu contain at least 4.42%, 2.12%, 0.68% and 0% of LAE, respectively. These results are in good agreement with those observed by TGA.

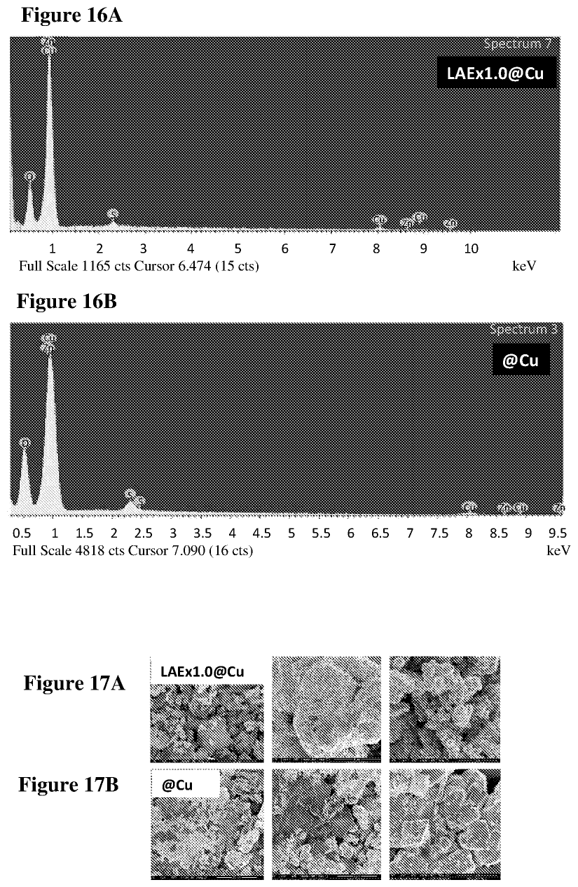

Energy dispersive spectroscopy (EDS): This analysis allows detection of elements on the surface of the tested sample using scanning electron microscopy (SEM). Therefore, this analysis allows to detect whether there are residues of zinc (Zn), sulfur (S) and oxygen (O) in the sample. FIG. 16 shows EDS results for composite LAE×1.0@Cu and for @Cu. In addition of copper, the two samples had low traces of Zn, S and O. The LAE×1.0@Cu composite contained Cu, O, S and Zn at 78.5%, 8.1%, 0.84 and 12.6%, respectively, while the @Cu sample, contained 51.0%, 16.2%, 2.2% and 30.6% of Cu, O, S and Zn, respectively. Based on this analysis, which assess only the surface of the tested sample, we cannot get valuable information regarding the presence and concentration of LAE in the LAE×1.0@Cu composite.

X-ray diffraction (XRD): This technique allows assessing information about the structure of the composite aggregates. This analysis revealed that part of the copper was oxidized to cuprite. In the LAE×1.0@Cu composite the percentages of Cu and cuprite were 87.1% and 12.9%, respectively, while in the @Cu sample, the percentages were 88.5% and 11.5%, respectively.

Scanning electron microscopy (SEM): This method provides valuable information regarding the physical structure and size of the composite particles. FIG. 17 shows representative images of the LAE×1.0@Cu composite and the @Cu sample. In both cases, the aggregate sizes were within the microscopic scale, with aggregate sizes varying from few to tens of micrometers. With that said, some differences were observed between the two samples. The LAE×1.0@Cu aggregates had lower density than the aggregates formed in the @Cu sample. In addition, most LAE×1.0@Cu aggregates have a quite circular shape, which was not characteristic of the aggregates formed in the @Cu samples (FIG. 17).

Characterization of the antimicrobial activity of the LAE@Cu composites: Antimicrobial activity of LAE: To assess the potential use of LAE@Cu composites for crop protection, the antimicrobial ability of LAE against several plant-pathogenic bacteria was determined first. This was done by determining the minimal inhibitory concentration (MIC) of LAE. These experiments revealed that LAE strongly inhibited growth of strains of Clavibacter michiganensis subsp. michiganensis (bacterial canker and wilt of tomato), Xanthomonas perforans (bacterial spot disease of tomato and pepper), Xanthomonas campestris pv. campestris (black rot disease of Brassicaceae plants) and Acidovorax citrulli (bacterial fruit blotch of cucurbit plants), with MIC values for these strains ranging between 5 and 16 μl/ml LAE.

Selection of A. citrulli for assessment of antimicrobial activity of LAE@Cu composites: Most experiments to characterize the antimicrobial activity of the composites were done with Acidovorax citrulli strain M6. Acidovorax citrulli (formerly Acidovorax avenae subsp. citrulli) is the causal agent of seedling blight and bacterial fruit blotch (BFB), a disease that threatens the cucurbit industry worldwide, and mainly of melon and watermelon. Strain M6 was isolated in Israel from a BFB outbreak in melon in 2002 and in the coming years became a model strain of A. citrulli for basic and applied research of BFB. Importantly, the management of BFB in the field strongly relies on the frequent use of Cu bactericides, yet with very limited efficiency. A. citrulli was also selected because from inventor's experience, this bacterium shows relatively high levels of tolerance to a variety of antimicrobial compounds, as compared with other plant-pathogenic bacterium. The results showing that A. citrulli is highly sensitive to LAE motivated us to select this bacterium for further characterization of the LAE@Cu composites.

Assessment of bactericidal activity of LAE @Cu composites in vitro: Bactericidal activity of LAE@Cu composites was tested by adding the composites to 50 ml-PBS suspensions containing ˜10⁷ colony forming units (CFU)/ml of A. citrulli M6. The suspensions were incubated at 30° C. with shaking for 30 min. Then, the concentrations of viable bacteria were determined following plating of serial dilutions on nutrient agar (NA, Difco) plates.

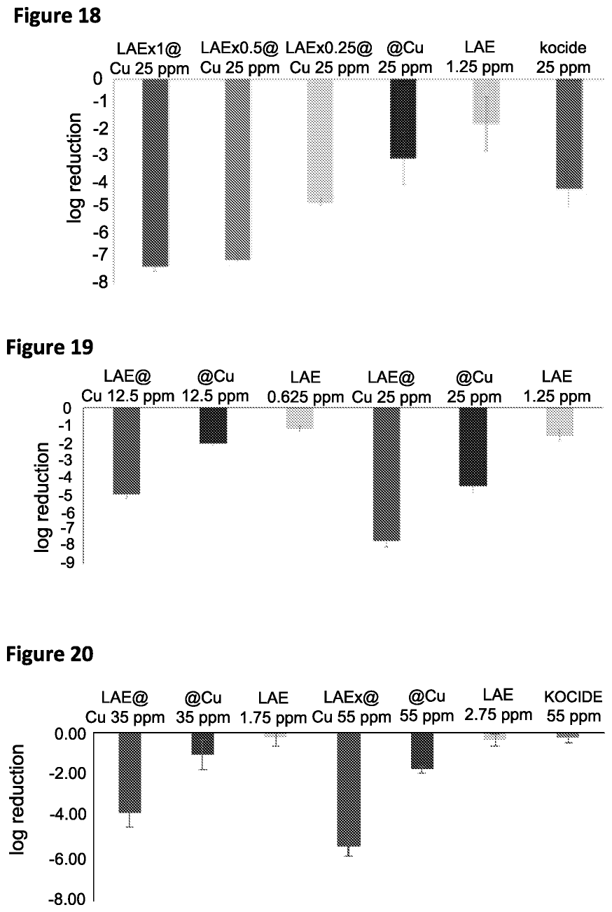

In first experiments the bactericidal activity of the three generated composites carrying different doping amounts of LAE-LAE×1.0@Cu, LAE×0.5@Cu and LAE×0.25@Cu- at concentration of 25 ppm was assessed. Controls included @Cu (composite-like compound without LAE) and a commercial bactericide, Kocide (DuPont), both at 25 ppm. An additional control was LAE at 1.25 ppm, which is the expected concentration of this molecule in 25 ppm of the LAE×1.0@Cu composite. The results of these experiments are shown in FIG. 18. The highest levels of bactericidal activity were exerted by the LAE×1.0@Cu and LAE×0.5@Cu. After 30 min of incubation, these treatments killed almost all bacteria, with a reduction of 7 logs of magnitude. The fact that these treatments had similar activity is not surprising since based on TGA (FIG. 14), these composites contain quite similar amounts of LAE (5 and 4.5% for LAE×1.0@Cu and LAE×0.5@Cu, respectively). The LAE×0.25@Cu composite, that based on TGA has ˜1% of LAE, also led to a significant reduction of the viable bacterial population, but significantly less than the two other composites (reduction of ˜5 logs of magnitude). Yet, the bactericidal activity of this composite was much higher than that measured for @Cu without LAE and was at the same level of the commercial bactericide Kocide. LAE at 1.25 ppm was able to reduce the A. citrulli population to 2 orders of magnitude. It is important to stress that this result means that this treatment still was able to kill most of the treated bacteria. Overall, these results support that the LAE and Cu in the form of LAE@Cu composites possess synergistic bactericidal activity.

In a second class of experiments, A. citrulli M6 cells were treated with two concentrations, 12.5 and 25 ppm, of the LAE×1.0@Cu composite. Controls were @Cu and LAE at the concentrations that corresponded to their expected concentrations in the composite treatments (12.5 ppm @Cu and 0.625 ppm LAE for the 12.5 ppm LAE×0.5@Cu treatment, and 25 ppm @Cu and 1.25 ppm LAE for the 25 ppm LAE×1.0@Cu treatment. As expected the bactericidal activity of 25 ppm LAE×1.0@Cu was significantly stronger than the activity of the same composite at 12.5 ppm (FIG. 19). These experiments also confirmed the synergistic interaction between Cu and LAE in the composite form.

In a third class of experiments, the bactericidal activity of different concentrations of the LAE×1.0@Cu composite (35 and 55 ppm) with Kocide at 55 ppm was compared. These experiments were carried out using a different plant-pathogenic bacterium, Xanthomonas perforans 97-2. These experiments, which are summarized in FIG. 20, strengthened the synergistic interaction between Cu and LAE in the composite form, as compared to individual application of these compounds. In addition, these experiments also revealed a clear advantage of the LAE×1.0@Cu composite over the commercial product Kocide, under tested conditions (FIG. 20).

Assessment of the ability of the LAE @Cu composites to reduce disease: To assess the potential of LAE@Cu composites for management of bacterial plant diseases, we carried out experiments using the A. citrulli-melon pathosystem. In these experiments, the foliage of three-week-old plants of melon (cv. AN-305; Origene Seeds) were sprayed with LAE1.0@Cu composites at a concentration of 200 ppm. Additional treatments included spraying with 200 ppm of @Cu, 10 ppm of LAE (corresponding to the concentration of LAE in 200 ppm LAE×1.0@Cu) and the commercial bactericide Kocide, which was applied at the recommended concentration for management of the disease (2 mg/ml). As controls, foliage were sprayed with double distilled water (DDW). After 4 h, the leaves were spray-inoculated with A. citrulli M6 at ˜10⁶ CFU/ml until run-off. The plants were kept in a growth chamber at 28° C., and a day:night regime of 14:10 h. During the first 24 h after inoculation the plants were covered with plastic bags to increase relative humidity and thus infection. Disease severity of inoculated leaves was determined 5 days after inoculation, using the following scale: 0, no symptoms; 1, few minor lesions; 2, few small necrotic spots; 3, increased necrotic spots in less than 25% of the leaf area; 4, increased necrotic spots in 25 to 50% of the leaf area; and 5, increased necrotic spots in more than 50% of the leaf area and dead leaf. The data were statistically analyzed by one-way analysis of variance (ANOVA) and Tukey's honest significant difference (HSD) test.

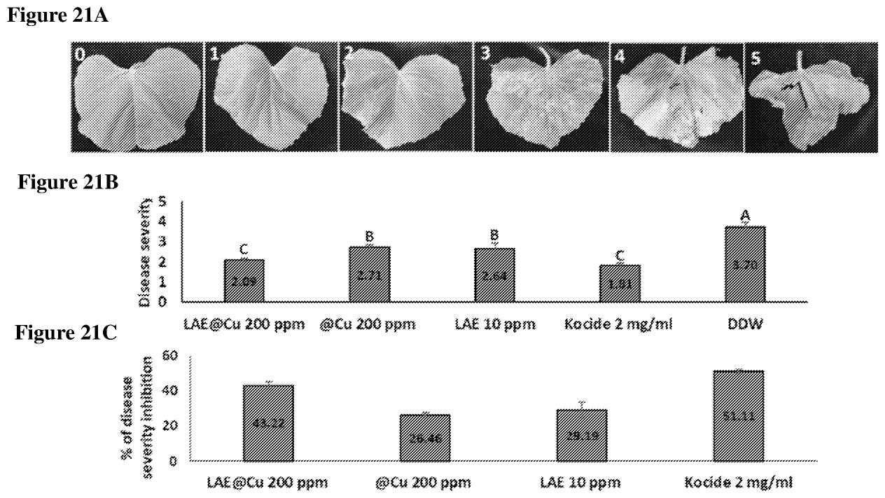

The results of three independent experiments that yielded similar results are summarized in FIG. 21. The LAE1.0@Cu composites were able to significantly (p<0.05) reduce disease severity as compared with non-treated controls, at a similar level than observed for the commercial bactericide Kocide.

It is important to emphasize that in these experiments, the concentration of Cu in the composites was much lower than its concentration in Kocide (190 vs. 1076 ppm, respectively).

Treatments with Cu (@[Cu]) and LAE alone also reduced the disease severity as compared with non-treated controls (26% and 29% respectively), but at a lower extent than the LAE1.0@[Cu] composites (FIG. 16C). These results show that the synergistic activity between Cu and LAE in the composites also occurs in planta. In conclusion, plant pathogenic bacteria are among the most important causal agents of plant diseases. Antimicrobial composites such as LAE×1.0@[Cu] have the potential to serve as novel crop protection agents. In this study, we showed that LAE×1.0@[Cu] composites have strong and synergistic antimicrobial activity in vitro and in planta. The aim of this report has been to proof of concept of the idea that molecules can be entrapped by metals and served as agriculture crop protection. In this research we currently characterized the mode of action of the composites, after revelling that the synergistic antimicrobial effect is more than the combination of LAE and Cu. Having the feasibility proof at hand, the scope of this methodology, its improvement, and its potential applications are next to be explored.

### Lauroyl Arginine Ethyl and Copper Composites

Materials and Methods

Bacterial strains and growth conditions: Bacterial strains used in this study: Acidovorax citrulli strain M6. Unless otherwise noted, cells were grown in modified Nutrient Broth (NB) medium (13 g/L) at 30° C. and stored at −80° C. in NB containing 25% glycerol. When we conducted killing assays we used PBS medium (8 g NaCl, 0.2 g KCl, 1.44 g Na2HPO4 and 0.24 g KH2PO4, pH 7.3)

Composites preparation methodology: For LAE@[Cu] preparation Copper sulfate (200 mg, 1.25×10-3 mol) was dissolved in 1 ml double distilled water (DDW). Zinc (Zn), the selected reducing agent, was added at an equal molar ratio as Cu (81 mg, 1.25×10-3 mol). After 30 sec, LAE was added at different concentrations (Table 5) and the solution was incubated at overnight at room temperature for 24 h. The resulting composites were then filtrated with a 0.22-μm membrane and dried under vacuum overnight.

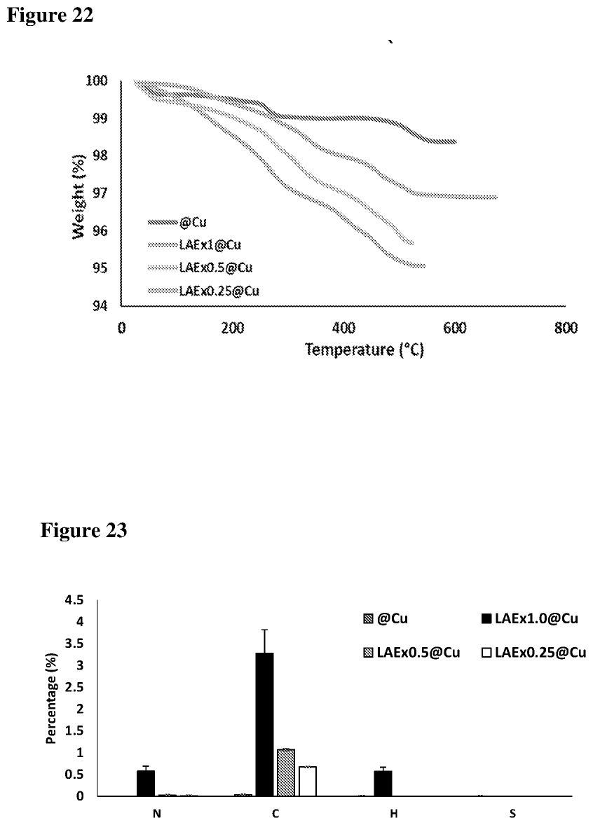

Bacterial killing assay: The bactericidal (killing) activity of LAE@[Cu] composites at a concentration of 25 ppm (1.25 mg in 50 ml), were tested in PBS containing ˜107 (CFU)/mL. The suspensions were incubated at 30° C. with shaking for 30 min. Then, the viable bacteria were determined following plating of serial dilutions on NA (nutrient agar) plates. Controls included @[Cu], the composite-like compound without LAE, and the commercial bactericide, Kocide®, at the same concentrations. An additional control was LAE at 1.25 ppm, which is the expected concentration of LAE in 25 ppm of the LAE×1.0@[Cu] composite as was observed by TGA analysis (FIG. 22).

Scanning electron microscopy (SEM): The images were taken in different magnification sizes, from ×60,000 to ×140,000 in a Jeol 7800 high resolution Scanning electron microscope. For taking these images with the bacteria after the killing assay, we took the bacteria and made fixation to them with 4% Glutaraldehyde buffer. We used small glass covered with poly lysin to paste the bacteria on the glass. Then, the samples were dried with ethanol and C.P.D chamber (K850 critical point drier), the ethanol replaced by liquid CO2 at 10° C. After that procedure the samples were coated with iridium and were ready to observe.

In-planta assay: Melon plants (cv. AN-305; Origene Seeds) were grown in a growth chamber at 28° C. (day:night, 14:10 h) in 10-cm diameter/12-cm height pots (three or four seedlings per pot) containing commercial soil for three weeks. The three youngest fully developed leaves of each plant were sprayed 200 ppm of LAE×1.0@[Cu] composites. Additional treatments included spraying 200 ppm of @[Cu], 10 ppm of LAE (corresponding to the entrapped concentration of LAE in 200 ppm LAE×1.0@[Cu]) and the commercial Cu-based commercial bactericide Kocide® 2000 (Gadot Agro; active compound: copper hydroxide), which was applied at the recommended concentration for management of the disease (2 mg/ml; 2000 ppm). As controls, the foliage was sprayed with double distilled water (DDW). After 4 h, the leaves were spray-inoculated with a suspension of A. citrulli M6 at ˜106 (CFU/mL) until run-off. During the first 24 h after inoculation the plants were covered with plastic bags to increase humidity and thus the infection. Disease severity was determined 5 days after inoculation.

Results

Percentage of the organic elements in three different doping concentrations of LAE (FIG. 23): LAE×1.0@[Cu], LAE×0.5@[Cu], LAE×0.25@[Cu] and @[Cu] were examined by elemental analyses. These results are averages and standard deviations of at least two repeats per sample.

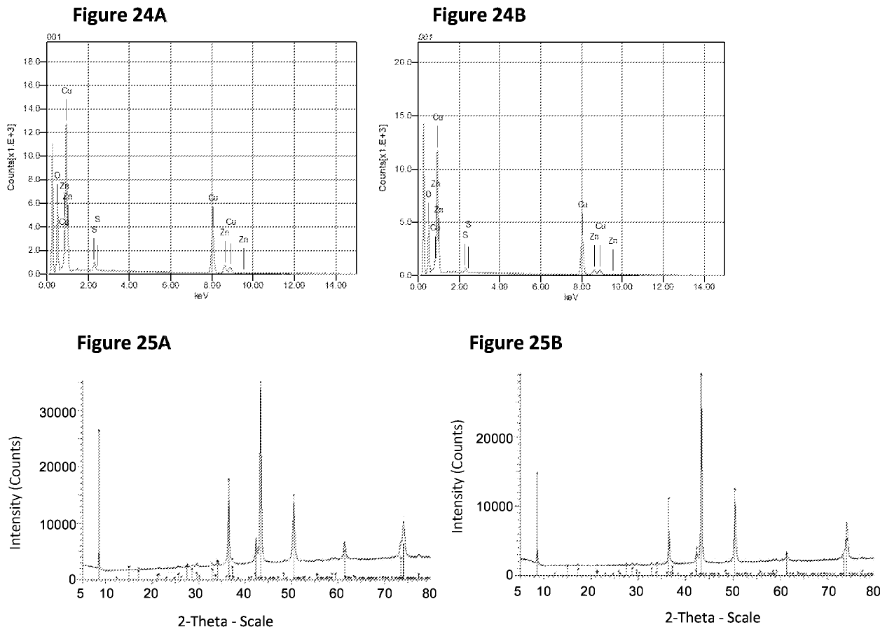

Energy dispersive spectroscopy (EDS): A. LAE×1.0@[Cu]. B. @[Cu]. Each spectrum represents the results of one analysis out of five independent analysis (with different batches) with similar results (FIG. 24).

X-ray diffraction (XRD): A. LAE×1.0@[Cu]. B. @[Cu]. Each spectrum represents the results of one analysis out of three independent analysis (with different batches) with similar results (FIG. 25).

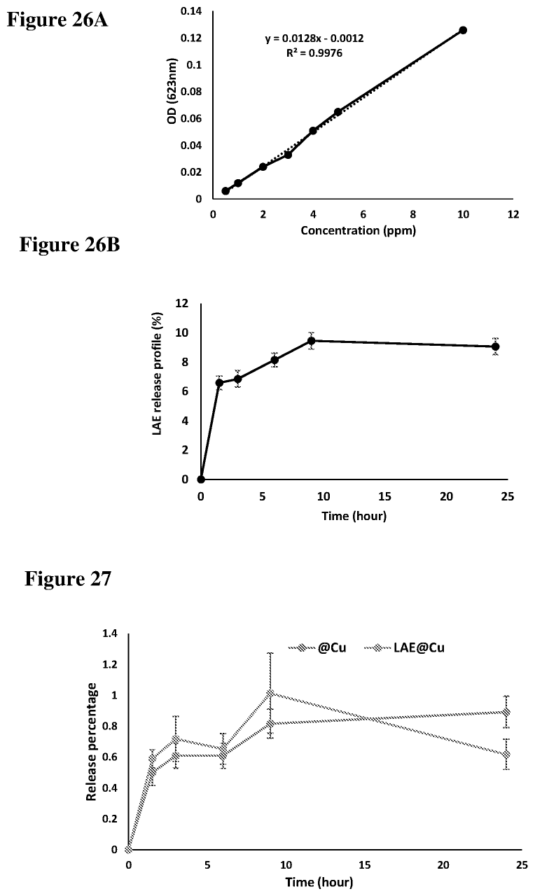

LAE release profile from LAEX1.0@[Cu]: 23 gr NH4SCN mixed with 11 gr Co(NO3)2 in 26 ml of double distilled water (DDW) to generate Co(SCN)4-2. LAE solution volume was 50 ml per each concentration. To every LAE solution 10 ml Co(SCN)4-2 was added and 5 ml 1,2 dichloroethane. All compounds were mixed and shaken for 5 minutes in a separatory funnel. After 5 minutes the two phases were separated. LAE-Co(SCN)4-2 complex was soluble at the organic phase (1,2 dichloroethane). 1 ml of this solution was taken for measurement (623 nm wavelength). A. Standard curve of LAE, different concentrations of LAE in DDW (0.5, 1, 2, 3, 4, 5 & 10 ppm) at 30° C., shaking conditions. B. LAE release profile from 400 ppm LAEX1.0@[Cu] (20 mg in 50 ml DDW), at 30° C., shaking conditions for different time intervals each (t=0, 1.5, 3, 6, 9 and 24 h) (Mean±SE, n=3) (FIG. 26).

Cu⁺2 release profile from LAEX1.0@[Cu] and @[Cu]: To assess the Cu+2 release profile from LAE×1.0@[Cu] and @[Cu] we used ICP-OES method. 400 ppm LAEX1.0@[Cu] or @[Cu] were measured and located in Erlenmeyer bottles (20 mg in 50 ml DDW), in a 30° C. shaking conditions. From each Erlenmeyer bottle containing LAE×1.0@[Cu] or @[Cu], a 1.5 ml sample was taken from the upper part of the liquid to Eppendorf tubes. Same volume of DDW was inserted to replace the taken sample. Results were taken during 24 h (Mean±SE, n=3). The samples were analyzed in the “Interdepartmental Equipment Unit” located in the Faculty of agriculture, food and environment (FIG. 27).

Random Peptide Mixtures Entrapped within a Copper-Cuprite Matrix as an Antimicrobial Agent Against Methicillin-Resistant Staphylococcus aureus Example:

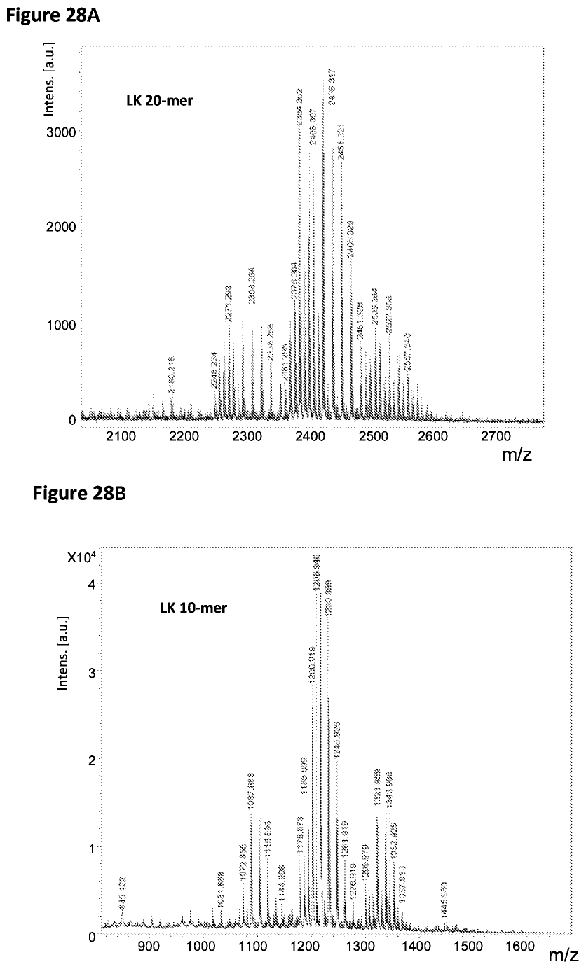

MALDI TOF MS representative spectra of leucine (L) lysine (K) random peptide mixtures after completing the synthesis is demonstrated in FIG. 28. The length is controlled and the mixtures contain a pool of peptides that share a similar molecular weight but have slightly different sequences.

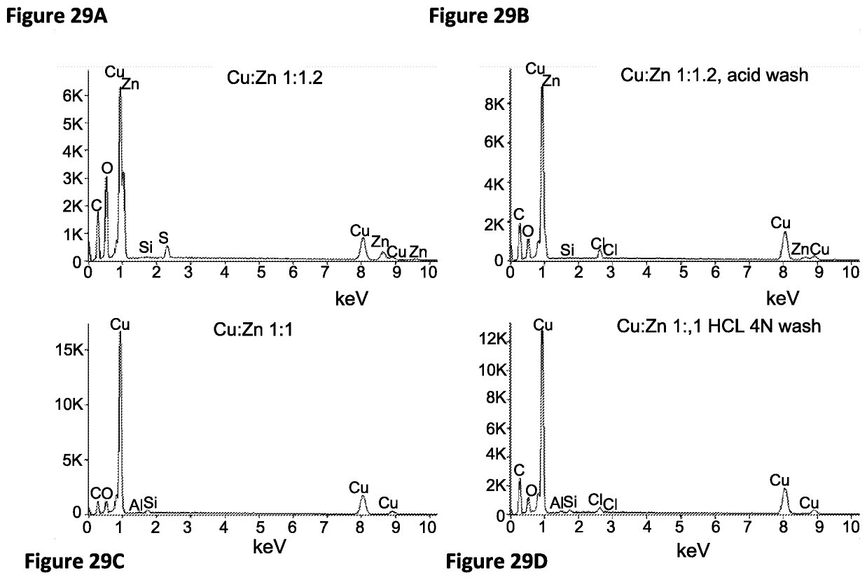

EDAX analysis of copper reduced by zinc at a ratio of 1:1 or 1:1.2 and with or without a 4N HCl acid wash is demonstrated in FIG. 29. Decreasing the molar ratio and washing with HCl reduces the presence of zinc in the composites.

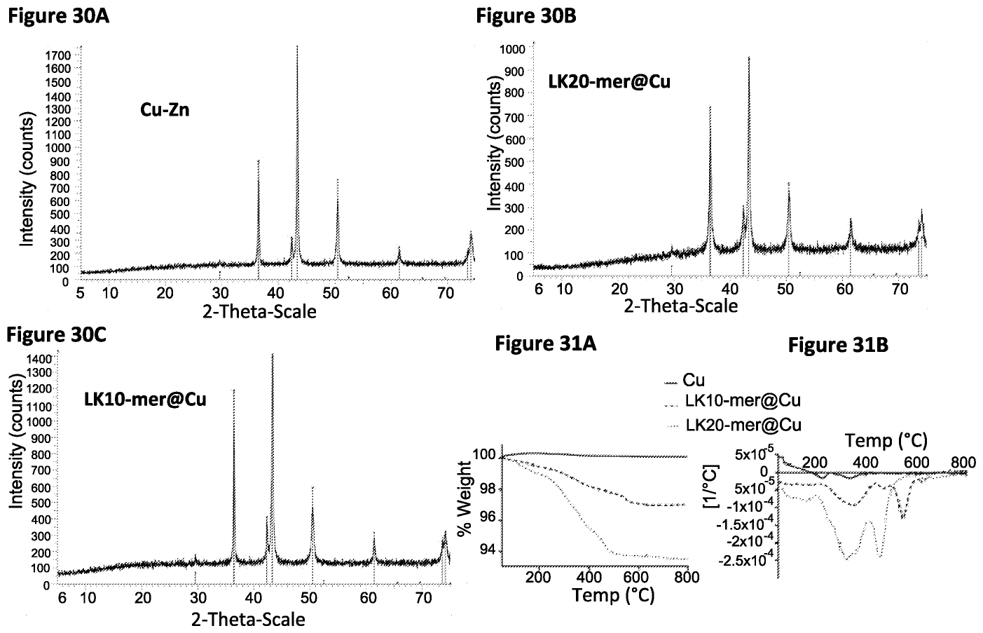

X-ray diffraction (XRD) results of composites are depicted in FIG. 30.

ICP results of three independent repeats are shown in Table 6. Ratio of copper ions released from [Cu] to copper ions released from LK20:mer@[Cu].

TABLE 6
ICP results.

| | [Cu]:LK20-mer@[Cu] |
|---|---|
| 1 | 1:1 |
| 2 | 0.94:1 |
| 3 | 0.85:1 |

The thermal gravity analysis of composites and first derivatives are shown in FIG. 31.

Examination of peptide release is presented in Table 7. Organic elemental analysis of composites before and after incubation in water, presented as percentage of weight.

TABLE 7
Examination of peptides release.

| | N | | C | | H | | S | |
|---|---|---|---|---|---|---|---|---|
| | AVG | SE | AVG | SE | AVG | SE | AVG | SE |
| [Cu] | 0.0026 | 0.0022 | 0.0331 | 0.0029 | 0.0000 | 0.0000 | 0.0037 | 0.0032 |
| [Cu] released | 0.0118 | 0.0072 | 0.0770 | 0.0138 | 0.0000 | 0.0000 | 0.0222 | 0.0124 |
| | 0.1231 | 0.0102 | 0.7446 | 0.0028 | 0.0593 | 0.0298 | 0.1007 | 0.0330 |
| LK10@[Cu] | | | | | | | | |
| LK10@[Cu] released | 0.1156 | 0.0117 | 0.9092 | 0.0080 | 0.0630 | 0.0315 | 0.0897 | 0.0450 |
| LK20@[Cu] | 0.2277 | 0.0117 | 1.1329 | 0.0357 | 0.2025 | 0.0064 | 0.1562 | 0.0439 |
| LK20@[Cu] released | 0.1031 | 0.0035 | 1.2962 | 0.0251 | 0.1069 | 0.0535 | 0.0879 | 0.0256 |

*Two independent experiments, 2 technical repeats for each.

1-113. (canceled)

114. A mixture of a plurality of conjugates, each conjugate comprising a fatty acid coupled to a peptide of 3 to 50 amino acid residues in length, wherein the conjugate peptides consist of hydrophobic and/or cationic amino acids, and wherein the ratio in the mixture of the total hydrophobic and cationic amino acids is optionally between 3:1 and 1:3.

115. The mixture according to claim 114, the mixture having at least one activity selected from the group consisting of antibacterial and antifungal activity.

116. The mixture according to claim 114, wherein the ratio is about 1:1.

117. The mixture according to claim 114, wherein the fatty acid is selected from saturated, unsaturated, monounsaturated and polyunsaturated fatty acids.

118-120. (canceled)

121. The mixture according to claim 114, wherein the hydrophobic amino acid is selected from the group consisting of phenylalanine, leucine, valine, alanine, isoleucine, and glycine.

122. (canceled)

123. The mixture according to claim 114, wherein the cationic amino acid is selected from the group consisting of lysine, arginine, histidine, ornithine, and di-amino butyric acid (Dab).

124. The mixture according to claim 114, wherein the hydrophobic amino acid is phenylalanine and the cationic amino acid is lysine.

125-128. (canceled)

129. A method of treating and/or preventing a plant disease, comprising administering to the plant an effective amount of a mixture according to claim 114.

130. The method according to claim 129, wherein the plant disease is caused by bacteria or fungi.

131. (canceled)

132. (canceled)

133. A pharmaceutical composition comprising the mixture of claim 114.

134. A method for treating an infection in a subject comprising administering to a subject in need thereof a therapeutically effective amount of the pharmaceutical composition of claim 133.

135. The method according to claim 134, wherein administering the pharmaceutical composition to the subject is selected from topical, intravenous, intraarterial, intramuscular, intraperitoneal, oral, ophthalmic, nasal, and intralesional administration.

136. The method according to claim 134, wherein the infection is caused by pathogenic organisms.

137-139. (canceled)

140. A disinfecting composition comprising a mixture according to claim 114.

141. A food preservative composition comprising a mixture according to claim 114.

142. A method for disinfecting an object comprising contacting the object with a disinfecting composition, the composition comprising as an active ingredient the mixture of claim 114.

143. (canceled)

144. (canceled)

145. The mixture according to claim 114, wherein two or more of said peptides having the same length, but differ in the selection of amino acids.

146. The mixture according to claim 114, wherein the number of peptides in the mixture is at least 2ⁿ, wherein n is the peptide length, and the number 2 designates peptides structured of two different amino acids.
